(19) United States
(12) Patent Application Publication
(10) Pub. No.: US 2023/0090925 A1
(43) Pub. Date: Mar. 23, 2023

(54) METHYLATION FRAGMENT PROBABILISTIC NOISE MODEL WITH NOISY REGION FILTRATION

(71) Applicant: GRAIL, LLC, Menlo Park, CA (US)

(72) Inventor: Qinwen Liu, Fremont, CA (US)

(21) Appl. No.: 17/946,460

(22) Filed: Sep. 16, 2022

Related U.S. Application Data
(60) Provisional application No. 63/246,030, filed on Sep. 20, 2021.

(51) Int. Cl. G16B 20/20; C12Q 1/6869; C12Q 1/6886

(52) U.S. Cl. CPC G16B 20/20; C12Q 1/6869; C12Q 1/6886; C12Q 2600/154; G16B 20/00

(57) ABSTRACT

A system and method are disclosed for training a cancer classifier. The method includes, for each training sample comprising a plurality of methylation sequence reads: for each methylation sequence read, applying a probabilistic noise model, corresponding to a genomic region of a plurality of genomics regions that the methylation sequence read overlaps with, to the methylation sequence read to determine an anomaly score indicating a likelihood of observing the methylation pattern in healthy samples. Each probabilistic noise model is trained with methylation sequence reads from healthy samples. The method includes determining a feature vector comprising a feature for each genomic region based on a count of methylation sequence reads overlapping the genomic region with an anomaly score below a threshold anomaly score. The method includes training the cancer classifier with the feature vectors of the training samples to determine a cancer prediction based on an input feature vector.

### CROSS-REFERENCE TO RELATED APPLICATION(S)

This application claims the benefit of and priority to U.S. Provisional Application No. 63/246,030 filed on Sep. 20, 2021, which is incorporated by reference in its entirety.

### BACKGROUND

### 1 Field of Art

This disclosure generally relates to a model for classifying nucleic acid fragments using methylation information.

### 2. Introduction

Analysis of circulating cell free nucleotides, such as cell free DNA (cfDNA) or cell free RNA (cfRNA), using next generation sequencing (NGS) is recognized as a valuable tool for detection and diagnosis of cancer or other diseases. Identifying rare variants indicative of cancer using NGS requires deep sequencing of nucleotide sequences from a biological sample such as a tissue biopsy or blood drawn from a subject. Detecting DNA that originated from tumor cells from a blood sample is difficult because circulating tumor DNA (ctDNA) or circulating tumor RNA (ctRNA) is typically present at low levels relative to other molecules in cfDNA extracted from the blood. The inability of existing methods to identify true positives (e.g., indicative of cancer in the subject) from signal noise diminishes the ability of known and future systems to distinguish true positives from false positives caused by noise sources, which can result in unreliable results for variant calling or other types of analyses. Moreover, errors introduced during sample preparation and sequencing can make accurate identification of rare variants difficult.

A number of different methods have been developed for detecting variants, such as single nucleotide variants (SNVs), in sequencing data. Most conventional methods have been developed for calling variants from DNA sequencing data obtained from a tissue sample. These methods may not be suitable for calling variants from deep sequencing data obtained from a cell free nucleotide sample.

For non-invasive diagnostic and monitoring of cancer, targeted sequencing data of cell free nucleotides serve as an important bio-source. However, detection of variants in deep sequencing data sets poses distinct challenges: the number of sequenced fragments tend to be several orders of magnitude larger (e.g., sequencing depth can be 2,000× or more), debilitating most of the existing variant callers in compute-time and memory usage.

### SUMMARY

A novel system and method are disclosed for determining anomalous methylation in nucleic acid fragments for use in detecting presence of a cancer state, a stage of cancer, another disease state, a tumor fraction, or some combination thereof. The method includes training probabilistic noise models that are parametrized per region of a plurality of regions of a genome. The probabilistic noise models are configured to input a methylation vector for a nucleic acid fragment and to output an anomaly score for the methylation vector.

Once the nucleic acid fragments are scored, the system labels one or more fragments as having an anomalous methylation pattern, or being anomalously methylated, if the anomaly score is above a threshold anomaly score. With the genomic regions used for classification, the system may determine features based on the anomalously methylated fragments, i.e., methylation sequence reads with anomalous methylation patterns. In one or more embodiments, the feature is a ratio of anomalously methylated fragments to the total number of fragments per region. The ratios can be used as features for training of a classifier for detecting presence of a cancer state, a stage of cancer, another disease state, a tumor fraction, or some combination thereof. In embodiments with weights, the feature derived for each region can be adjusted according to the weight set for each respective region.

The system may filter one or more regions from use in classification having an above-threshold percentage of White Blood Cell (WBC) samples having anomalously methylated fragments overlap those regions. The system effectively filters out regions that are particularly noisy for hematological conditions relating to white blood cells, such as various types of leukemia and lymphoma, to improve sensitivity in detecting other cancer or disease types. The system may perform a similar process of region filtration with other cancer types, e.g., breast cancer. In one or more embodiments, the system may weight the noisy regions with a lower weight rather than excluding them completely from the classification. In one or more embodiments, a region-weighting scheme assigns a weight based on a percentage of WBC samples having anomalous fragments overlapping the genomic region. The genomic region-weighting scheme could be rule-based, e.g., a region with above 40% of samples having an overlapping anomalous fragment is weighted as 0, a region between 30% and 40% of samples is weighted as 0.2, a region between 20% and 30% of samples is weighted as 0.4, while a region below 20% of samples is weighted as 1. In one or more other embodiments, the weights can be adaptively adjusted based on performance of a downstream classifier.

In a first aspect of the disclosure, a method for training a cancer classifier comprises: training a probabilistic noise model parameterized by, for each genomic region of a plurality of genomic regions of a genome, a mean and a dispersion of a measure of methylated CpG sites in a first plurality of methylation sequence reads from healthy samples; for each training sample, determining an anomaly score for each of a plurality of methylation sequence reads from the training sample by applying a trained probabilistic noise model associated with the genomic region that the methylation sequence read overlaps with; for each training sample, determining a count of anomalously methylated fragments in each genomic region of the plurality of genomic regions by comparing the anomaly scores of the methylation sequence reads with a threshold anomaly score; for each training sample, determining a ratio, for each genomic region of the plurality of genomic regions, of the count of anomalously methylated fragments in the genomic region to a total number of methylation sequence reads in the genomic region; for each training sample, generating a feature vector comprising the ratios over the plurality of genomic regions; and training a classifier to determine a cancer prediction using the feature vectors of the training samples.

According to the first aspect, training the probabilistic noise model comprises: determining posterior distributions of the mean and the dispersion for each genomic region of the plurality of genomic regions using a Bayesian inference, wherein the Bayesian inference is determined using Markov chain Monte Carlo.

According to the first aspect, the posterior distributions are beta binomial distributions.

According to the first aspect, the anomaly score is determined by the trained probabilistic noise models for each methylation sequence read is based on a p-value for the methylation sequence read indicating a probability that the methylation sequence read is anomalously methylated.

According to the first aspect, the anomaly score for each methylation sequence read is the p-value for the methylation sequence read.

According to the first aspect, the anomaly score for each methylation sequence read is determined by applying a transformation to the p-value determined for the methylation sequence read.

According to the first aspect, the transformation is a logarithmic or nonlinear function.

According to the first aspect, a first genomic region of the plurality of genomic regions is associated with a first mean and a first dispersion, and wherein a second genomic region of the plurality of genomic regions is associated with a second mean and a second dispersion different than the first mean and the first dispersion, respectively.

According to the first aspect, a first genomic region of the plurality of genomic regions includes a first number of CpG sites, and the second genomic region of the plurality of genomic regions includes a second number of CpG sites, that is different than the first number of CpG sites.

According to the first aspect, the method further comprises: obtaining a test sample from an individual; generating a plurality of sequence reads from a second plurality of methylation sequence reads of the test sample; determining an anomaly score for each of the second plurality of methylation sequence reads of the test sample by applying the trained probabilistic noise model associated with the genomic region that the methylation sequence read overlaps with; determining a count of anomalously methylated fragments in each genomic region of the plurality of genomic regions by comparing the anomaly scores of the methylation sequence reads of the test sample with the threshold anomaly score; determining a ratio, for each genomic region of the plurality of genomic regions, of the count of anomalously methylated fragments of the test sample in the genomic region to a total number of methylation sequence reads of the test sample in the genomic region; and generating a test feature vector comprising the ratios for the test sample over the plurality of genomic regions; determining a cancer prediction for the test sample by applying the trained classifier to the test feature vector.

According to the first aspect, the cancer prediction estimates a tumor fraction of the test sample.

According to the first aspect, the cancer prediction indicates a presence of a disease state in the test sample.

According to the first aspect, the disease state is selected from the group consisting of breast cancer, uterine cancer, cervical cancer, ovarian cancer, bladder cancer, urothelial cancer of renal pelvis, renal cancer other than urothelial, prostate cancer, anorectal cancer, colorectal cancer, esophageal cancer, gastric cancer, hepatobiliary cancer arising from hepatocytes, hepatobiliary cancer arising from cells other than hepatocytes, pancreatic cancer, squamous cell cancer of the upper gastrointestinal tract, upper gastrointestinal cancer other than squamous, head and neck cancer, lung cancer, lung adenocarcinoma, small cell lung cancer, squamous cell lung cancer and cancer other than adenocarcinoma or small cell lung cancer, neuroendocrine cancer, melanoma, thyroid cancer, sarcoma, multiple myeloma, lymphoma, and leukemia, and other hematological conditions.

According to the first aspect, the cancer prediction indicates a stage of cancer present in the test sample.

According to the first aspect, the methylation sequence reads include methylation information of cell-free DNA fragments.

According to the first aspect, the method further comprises: for each White Blood Cell (WBC) sample of a plurality of WBC samples, determining an anomaly score for each of a plurality of methylation sequence reads from the WBC sample by applying the trained probabilistic noise model associated with the genomic region that the methylation sequence read overlaps with; for each WBC sample, determining a count of anomalously methylated fragments in each genomic region of the plurality of genomic regions by comparing the anomaly scores of the methylation sequence reads with a threshold anomaly score; and for each genomic region of the plurality of genomic regions, labeling the genomic region as noisy if there is more than a threshold percentage of WBC samples with a threshold number of anomalously methylated fragments overlapping the genomic region.

According to the first aspect, the method further comprises: excluding the genomic regions labeled as noisy from use in the training of the classifier, wherein the feature vectors generated for the training samples exclude the ratios of the genomic regions labeled as noisy.

According to the first aspect, the method further comprises: assigning a default weight to each genomic region of the plurality of genomic regions; reassigning a first weight to the genomic regions labeled as noisy, wherein the first weight is lower than the default weight; and for each training sample, multiplying each ratio of the feature vector with the corresponding weight for the genomic region associated with the ratio.

According to the first aspect, the threshold percentage is selected from the range of 5% to 40%.

According to the first aspect, the threshold number of anomalously methylated fragments is selected from the range of 1-10.

According to a second aspect, a method for training a cancer classifier comprises: for each of a plurality of training samples comprising cancer samples and non-cancer samples, each training sample comprising a plurality of methylation sequence reads including methylation information of cell-free DNA fragments: for each methylation sequence read, applying a probabilistic noise model, corresponding to a genomic region of a plurality of genomics regions that the methylation sequence read overlaps with, to the methylation sequence read to determine an anomaly score indicating a likelihood of observing the methylation pattern in healthy samples, wherein each probabilistic noise model is trained with methylation sequence reads from healthy samples; determining a feature vector comprising a feature for each genomic region based on a count of methylation sequence reads overlapping the genomic region with an anomaly score below a threshold anomaly score; and training the cancer classifier with the feature vectors of the training samples to determine a cancer prediction based on an input feature vector.

According to the second aspect, each probabilistic noise model is parametrized by a mean and a dispersion of a measure of methylated CpG sites in methylation sequence reads from the healthy samples.

According to the second aspect, each probabilistic noise model is trained by: determining posterior distributions of the mean and the dispersion for each genomic region of the plurality of genomic regions using a Bayesian inference, wherein the Bayesian inference is determined using Markov chain Monte Carlo.

According to the second aspect, the posterior distributions are beta binomial distributions.

According to the second aspect, the anomaly score determined by the trained probabilistic noise models for each methylation sequence read is based on a p-value for the methylation sequence read indicating a probability that the methylation sequence read is anomalously methylated.

According to the second aspect, the anomaly score for each methylation sequence read is the p-value for the methylation sequence read.

According to the second aspect, the anomaly score for each methylation sequence read is determined by applying a transformation to the p-value determined for the methylation sequence read.

According to the second aspect, the transformation is a logarithmic or nonlinear function.

According to the second aspect, a first genomic region of the plurality of genomic regions is associated with a first mean and a first dispersion, and wherein a second genomic region of the plurality of genomic regions is associated with a second mean and a second dispersion different than the first mean and the first dispersion, respectively.

According to the second aspect, a first genomic region of the plurality of genomic regions includes a first number of CpG sites, and the second genomic region of the plurality of genomic regions includes a second number of CpG sites, that is different than the first number of CpG sites.

According to the second aspect, the method further comprises: for each White Blood Cell (WBC) sample of a plurality of WBC samples, determining an anomaly score for each of a plurality of methylation sequence reads from the WBC sample by applying the trained probabilistic noise model associated with the genomic region that the methylation sequence read overlaps with; for each WBC sample, determining a count of anomalously methylated fragments in each genomic region of the plurality of genomic regions by comparing the anomaly scores of the methylation sequence reads with a threshold anomaly score; and for each genomic region of the plurality of genomic regions, labeling the genomic region as noisy if there is more than a threshold percentage of WBC samples with a threshold number of anomalously methylated fragments overlapping the genomic region.

According to the second aspect, the method further comprises: excluding the genomic regions labeled as noisy from use in the training of the classifier, wherein the feature vectors generated for the training samples exclude the ratios of the genomic regions labeled as noisy.

According to the second aspect, the method further comprises: assigning a default weight to each genomic region of the plurality of genomic regions; reassigning a first weight to the genomic regions labeled as noisy, wherein the first weight is lower than the default weight; and for each training sample, multiplying each ratio of the feature vector with the corresponding weight for the genomic region associated with the ratio.

According to the second aspect, the threshold percentage is selected from the range of 5% to 40%.

According to the second aspect, the threshold number of anomalously methylated fragments is selected from the range of 1-10.

According to a third aspect, a method, for predicting cancer status of a test sample comprising a plurality of methylation sequence reads including methylation information of cell-free DNA fragments, comprises: for each methylation sequence read, applying a probabilistic noise model, corresponding to a genomic region of a plurality of genomics regions that the methylation sequence read overlaps with, to the methylation sequence read to determine an anomaly score indicating a likelihood of observing the methylation pattern in healthy samples, wherein each probabilistic noise model is trained with methylation sequence reads from healthy samples; determining a feature vector comprising a feature for each genomic region based on a count of methylation sequence reads overlapping the genomic region with an anomaly score below a threshold anomaly score; and applying a cancer classifier to the feature vector to determine a cancer prediction.

According to the third aspect, the cancer classifier is trained by the method of the first or second aspect.

According to the third aspect, the cancer prediction estimates a tumor fraction of the test sample.

According to the third aspect, the cancer prediction indicates a presence of a disease state in the test sample.

According to the third aspect, the disease state is selected from the group consisting of breast cancer, uterine cancer, cervical cancer, ovarian cancer, bladder cancer, urothelial cancer of renal pelvis, renal cancer other than urothelial, prostate cancer, anorectal cancer, colorectal cancer, esophageal cancer, gastric cancer, hepatobiliary cancer arising from hepatocytes, hepatobiliary cancer arising from cells other than hepatocytes, pancreatic cancer, squamous cell cancer of the upper gastrointestinal tract, upper gastrointestinal cancer other than squamous, head and neck cancer, lung cancer, lung adenocarcinoma, small cell lung cancer, squamous cell lung cancer and cancer other than adenocarcinoma or small cell lung cancer, neuroendocrine cancer, melanoma, thyroid cancer, sarcoma, multiple myeloma, lymphoma, and leukemia, and other hematological conditions.

According to the third aspect, the cancer prediction indicates a stage of cancer present in the test sample.

According to the third aspect, the method further comprises: returning the cancer prediction with treatment recommendation based on the cancer prediction.

According to a fourth aspect, a method, for training a plurality of probabilistic noise models, comprises: for each genomic region of a plurality of genomic regions: aggregating methylation sequence reads from healthy samples overlapping the genomic region, each healthy sample comprising a plurality of methylation sequence reads including methylation information of cell-free DNA fragments; training a probabilistic noise model with the aggregated methylation sequence reads, wherein the trained probabilistic noise model is configured to input a methylation sequence read and output an anomaly score indicating a likelihood of observing the methylation pattern in healthy samples.

According to the fourth aspect, training the probabilistic noise model comprises: determining posterior distributions of the mean and the dispersion for the genomic region using a Bayesian inference determined using Markov chain Monte Carlo.

According to the fourth aspect, the posterior distributions are beta binomial distributions.

According to the fourth aspect, the anomaly score determined by the trained probabilistic noise models for each methylation sequence read is based on a p-value for the methylation sequence read indicating a probability that the methylation sequence read is anomalously methylated.

According to the fourth aspect, the anomaly score for each methylation sequence read is the p-value for the methylation sequence read.

According to the fourth aspect, the anomaly score for each methylation sequence read is determined by applying a transformation to the p-value determined for the methylation sequence read.

According to the fourth aspect, the transformation is a logarithmic or nonlinear function.

According to the fourth aspect, a first genomic region of the plurality of genomic regions is associated with a first mean and a first dispersion, and wherein a second genomic region of the plurality of genomic regions is associated with a second mean and a second dispersion different than the first mean and the first dispersion, respectively.

According to the fourth aspect, a first genomic region of the plurality of genomic regions includes a first number of CpG sites, and the second genomic region of the plurality of genomic regions includes a second number of CpG sites, that is different than the first number of CpG sites.

According to the fourth aspect, each genomic region is no greater than 50, no greater than 60, no greater than 70, no greater than 80, no greater than 90, or no greater than 100 CpG sites.

According to the fourth aspect, each genomic region in the plurality of regions comprises at least 1, at least 2, at least 3, at least 4, at least 5, at least 6, at least 7, at least 8, at least 9, at least 10, at least 20, at least 30, or more than 30 CpG sites.

According to the fourth aspect, each genomic region comprises one or more adjacent CpG sites.

According to a fifth aspect, a system comprises a computer processor and a memory, the memory storing computer program instructions that, when executed by the computer processor, cause the processor to perform the method of any of the above aspects.

According to a sixth aspect, a non-transitory computer-readable medium stores computer program instructions which, when executed by an electronic device including a processor, cause the device to perform the method of any of the above aspects.

According to a seventh aspect, a computer program product comprises a non-transitory computer-readable medium storing a machine-learning cancer classifier for predicting cancer in a test sample, wherein the product is made by the method of the first or second aspect.

According to an eight aspect, a computer program product comprises a non-transitory computer-readable medium storing a plurality of probabilistic noise models for determining anomalously methylated methylation sequence reads, wherein the product is made by the method of the fourth aspect.

According to a ninth aspect, a treatment kit comprises: reagents for isolating DNA fragments from a test sample and sequencing the isolated DNA fragments to obtain a plurality of methylation sequence reads including methylation information of the DNA fragments;

instructions for using the reagents; and a non-transitory computer-readable storage medium storing instructions for analyzing the methylation sequence reads, the instructions that, when executed by a processor, cause the processor to perform operations comprising: for each methylation sequence read, applying a probabilistic noise model, corresponding to a genomic region of a plurality of genomics regions that the methylation sequence read overlaps with, to the methylation sequence read to determine an anomaly score indicating a likelihood of observing the methylation pattern in healthy samples, wherein each probabilistic noise model is trained with methylation sequence reads from healthy samples; determining a feature vector comprising a feature for each genomic region based on a count of methylation sequence reads overlapping the genomic region with an anomaly score below a threshold anomaly score; applying a cancer classifier to the feature vector to determine a cancer prediction; and returning the cancer prediction with treatment recommendation based on the cancer prediction.

According to the ninth aspect, the cancer classifier is trained by the method of the first or second aspect.

According to the ninth aspect, the plurality of probabilistic models is trained by the method of the fourth aspect.

### BRIEF DESCRIPTION OF DRAWINGS

FIG. 1 is an exemplary flowchart describing an overall workflow of cancer classification of a sample, according to one or more embodiments.

FIG. 2A illustrates a flowchart of devices for sequencing nucleic acid samples according to an embodiment.

FIG. 2B illustrates a block diagram of an analytics system, according to an embodiment.

FIG. 3 is a flowchart describing a process of sequencing nucleic acids, according to some embodiments.

FIG. 4 is an illustration of a part of the process of FIG. 3 of sequencing nucleic acids fragments to obtain methylation status at one or more methylation sites, according to some embodiments.

FIG. 5A is a flowchart of a method for training one or more probabilistic noise models.

FIG. 5B is a flowchart of a method for utilizing the trained probabilistic noise models, according to some embodiments.

FIG. 6A is a flowchart of a method for training a classifier to determine a cancer prediction from nucleic acid fragments of a sample, according to some embodiments.

FIG. 6B is a flowchart of a method for determining a cancer prediction for a test sample, according to some embodiments.

FIG. 7 illustrates posterior distributions of parameters of a probabilistic noise model, according to example implementations.

FIGS. 8A, 8B, and 8C illustrate fractions of fragment methylation and counts of methylated CpG sites, according to example implementations.

FIGS. 9A and 9B illustrate mean and dispersion parameter estimation using simulations of varying sample size, according to example implementations.

FIG. 10A illustrates cumulative frequency of anomalously methylated fragments by disease state, according to example implementations.

FIG. 10B illustrates cumulative frequency of anomalously methylated fragments by cancer stage, according to example implementations.

FIG. 11 illustrates a receiver operating characteristic (ROC) curve indicating performance of a trained classifier for detecting anomalously methylated fragments, according to example implementations.

FIG. 12 illustrates a table the detection rates of classifiers, some classifiers trained with filtered noisy regions, according to example implementations.

FIG. 13 shows a schematic of an example computer system for implementing various methods of the present invention.

The figures depict embodiments of the present invention for purposes of illustration only. One skilled in the art will readily recognize from the following discussion that alternative embodiments of the structures and methods illustrated herein can be employed without departing from the principles of the invention described herein.

### DETAILED DESCRIPTION

Reference will now be made in detail to several embodiments, examples of which are illustrated in the accompanying figures. It is noted that wherever practicable similar or like reference numbers may be used in the figures and may indicate similar or like functionality.

### I. Overview

### I.A. Cancer Classification Workflow

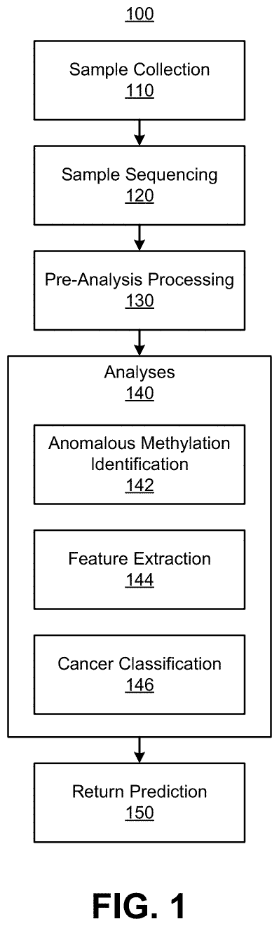

FIG. 1 is an exemplary flowchart describing an overall workflow 100 of cancer classification of a sample, according to one or more embodiments. The workflow 100 is by one or more entities, e.g., including a healthcare provider, a sequencing device, an analytics system, etc. Objectives of the workflow include detecting and/or monitoring cancer in individuals. From a healthcare standpoint, the workflow 100 can serve to supplement other existing cancer diagnostic tools. The workflow 100 may serve to provide early cancer detection and/or routine cancer monitoring to better inform treatment plans for individuals diagnosed with cancer. The overall workflow 100 may include additional/fewer steps than those shown in FIG. 1.

A healthcare provider performs sample collection 110. An individual to undergo cancer classification visits their healthcare provider. The healthcare provider collects the sample for performing cancer classification. Examples of biological samples include, but are not limited to, tissue biopsy, blood, whole blood, plasma, serum, urine, cerebrospinal fluid, fecal, saliva, sweat, tears, pleural fluid, pericardial fluid, or peritoneal fluid of the subject. The sample includes genetic material belonging to the individual, which may be extracted and sequenced for cancer classification. Once the sample is collected, the sample is provided to a sequencing device. Along with the sample, the healthcare provider may collect other information relating to the individual, e.g., biological sex, age, ethnicity, smoking status, any prior diagnoses, etc.

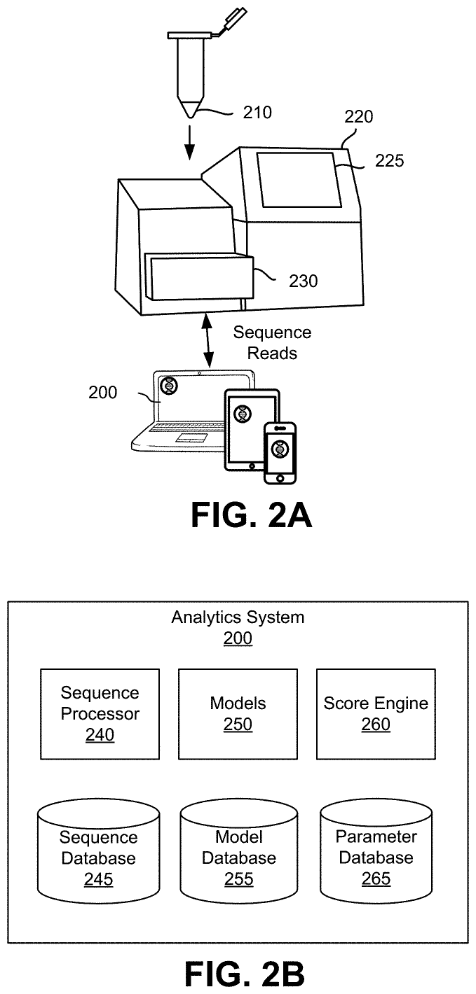

A sequencing device performs sample sequencing 120. An example sequencing device is described in FIG. 2A. A lab clinician may perform one or more processing steps to the sample in preparation of sequencing. Once prepared, the clinician loads the sample in the sequencing device. The sequencing device generally extracts and isolates fragments of nucleic acid that are sequenced to determine a sequence of nucleobases corresponding to the fragments. Sequencing may also include amplification of nucleic material. Different sequencing processes include Sanger sequencing, fragment analysis, and next-generation sequencing. Sequencing may be whole-genome sequencing or targeted sequencing with a target panel. In context of DNA methylation, bisulfite sequencing (e.g., further described in FIGS. 2A & 2B) can determine methylations status through bisulfite conversion of unmethylated cytosines at CpG sites. Sample sequencing 120 yields sequences for a plurality of nucleic acid fragments in the sample. In one or more embodiments, the sequences may include methylation state vectors, wherein each methylation state vector describes the methylation statuses for CpG sites on a fragment.

An analytics system performs pre-analysis processing 130. An example analytics system is described in FIG. 2B. Pre-analysis processing 130 may include, but not limited to, de-duplication of sequence reads, determining metrics relating to coverage, determining whether the sample is contaminated, removal of contaminated fragments, calling sequencing error, etc.

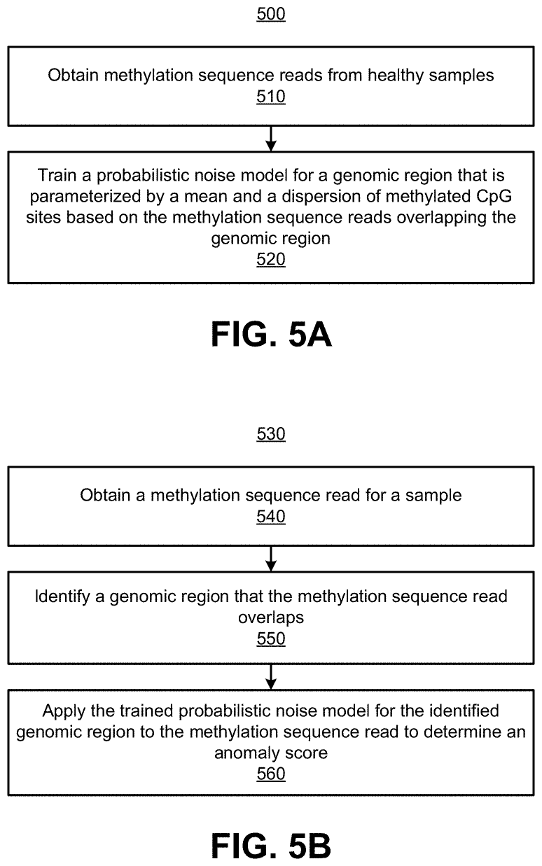

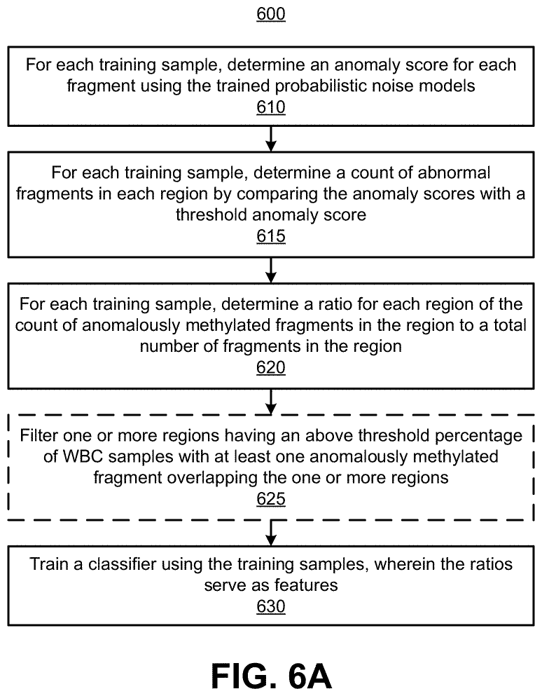

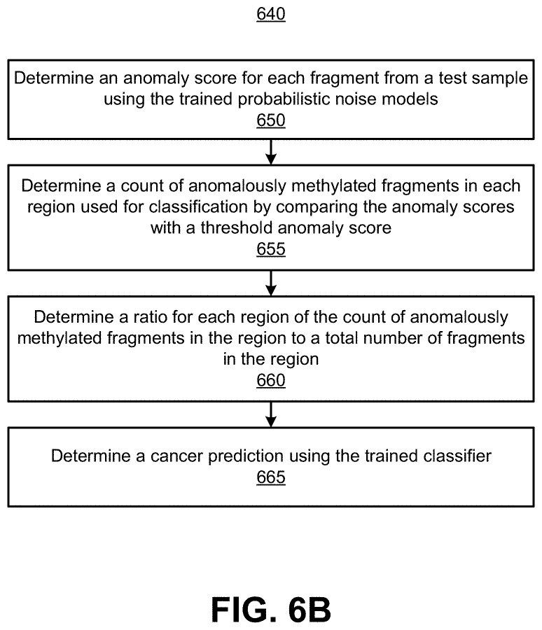

The analytics system performs one or more analyses 140. The analyses are statistical analyses or application of one or more trained models to predict at least a cancer status of the individual from whom the sample is derived. Different genetic features may be evaluated and considered, such as methylation of CpG sites, single nucleotide polymorphisms (SNPs), insertions or deletions (indels), other types of genetic mutation, etc. In context of methylation, analyses 140 may include anomalous methylation identification 142 (e.g., further described in FIGS. 5A & 5B), feature extraction 144 (e.g., further described in FIGS. 6A & 6B), and applying a cancer classifier 146 to determine a cancer prediction (e.g., further described in FIGS. 6A & 6B). The cancer classifier 146 inputs the extracted features to determine a cancer prediction. The cancer prediction may be a label or a value. The label may indicate a particular cancer state, e.g., binary labels can indicate presence or absence of cancer, multiclass labels can indicate one or more cancer types from a plurality of cancer types that are screened for. The value may indicate a likelihood of a particular cancer state, e.g., a likelihood of cancer, and/or a likelihood of a particular cancer type.

The analytics system returns the prediction 150 to the healthcare provider. The healthcare provider may establish or adjust a treatment plan based on the cancer prediction. Optimization of treatment is further described in Section IV.C. Treatment.

The cancer classification workflow 100 is a technical and novel solution to the field of early cancer detection. In early cancer detection, tumor cells have just begun to or have yet to develop, slipping under the traditional tools for cancer detection. These traditional techniques heavily rely on advanced imaging techniques to spot significant growths or lesions to further inspect through biopsy and sequencing. These techniques are ill equipped for the problem of attempting to detect cancer in pre-development or early development. The cancer classification workflow 100 provides for a technical solution by surveying an individual's genetic material to detect genetic signatures or features that are indicative of cancer or the imminent onset thereof. Even still, identifying the genetic features is a labor-intensive task and akin to finding a needle in a haystack. The training and utilization of probabilistic models, as will be further described below, embodies a technical solution to determine anomalously methylated fragments in a test sample, that may comprise more than 10,000 unique nucleic acid molecules. The analytics system then utilizes the anomalously methylated fragments, those needles in the haystack, to train a cancer classifier that can detect cancer signal to a high degree of confidence. Such detection of cancer signal can be practically applied for early cancer detection, and other practical applications, such as monitoring the efficacy of a cancer treatment. Moreover, these analytical techniques do not embody abstract ideas as they are grounded in analyzing sequence reads of physical nucleic acid fragments present in a biological sample collected from a living individual.

### I.B. Methylation Overview

In accordance with the present description, cfDNA fragments from an individual are treated, for example by converting unmethylated cytosines to uracils, sequenced and the sequence reads compared to a reference genome to identify the methylation states at specific CpG sites within the DNA fragments. Each CpG site may be methylated or unmethylated. Identification of anomalously methylated fragments, in comparison to healthy individuals, may provide insight into a subject's cancer status. DNA methylation anomalies (compared to healthy controls) can cause different effects, which may contribute to cancer. Various challenges arise in the identification of anomalously methylated cfDNA fragments. First off, determining a DNA fragment to be anomalously methylated can hold weight in comparison with a group of control individuals, such that if the control group is small in number, the determination loses confidence due to statistical variability within the smaller size of the control group. Additionally, among a group of control individuals, methylation status can vary which can be difficult to account for when determining a subject's DNA fragments to be anomalously methylated. On another note, methylation of a cytosine at a CpG site can causally influence methylation at a subsequent CpG site. To encapsulate this dependency can be another challenge in itself.

Methylation can typically occur in deoxyribonucleic acid (DNA) when a hydrogen atom on the pyrimidine ring of a cytosine base is converted to a methyl group, forming 5-methylcytosine. In particular, methylation can occur at dinucleotides of cytosine and guanine referred to herein as “CpG sites”. In other instances, methylation may occur at a cytosine not part of a CpG site or at another nucleotide that is not cytosine; however, these are rarer occurrences. In this present disclosure, methylation is discussed in reference to CpG sites for the sake of clarity. Anomalous DNA methylation can be identified as hypermethylation or hypomethylation, both of which may be indicative of cancer status. Throughout this disclosure, hypermethylation and hypomethylation can be characterized for a DNA fragment, if the DNA fragment comprises more than a threshold number of CpG sites with more than a threshold percentage of those CpG sites being methylated or unmethylated.

The principles described herein can be equally applicable for the detection of methylation in a non-CpG context, including non-cytosine methylation. In such embodiments, the wet laboratory assay used to detect methylation may vary from those described herein. Further, the methylation state vectors discussed herein may contain elements that are generally sites where methylation has or has not occurred (even if those sites are not CpG sites specifically). With that substitution, the remainder of the processes described herein can be the same, and consequently the inventive concepts described herein can be applicable to those other forms of methylation.

### I.C. Definitions

The term “individual” refers to a human, an animal, or any other multicellular living organism. The term “healthy individual” refers to an individual presumed to not have a cancer or disease. The term “subject” refers to an individual who is known to have, or potentially has, a cancer or disease.

The term “sequence reads” refers to nucleotide sequences read from a sample obtained from an individual. Sequence reads can be obtained through various methods known in the art. The term “methylation sequence read” may further refer to any nucleotide sequences that reveal methylation information of the nucleic acid fragment, e.g., that may be treated via bisulfite sequencing.

The term “read segment” or “read” refers to any nucleotide sequences including sequence reads obtained from an individual and/or nucleotide sequences derived from the initial sequence read from a sample obtained from an individual. For example, a read segment can refer to an aligned sequence read, a collapsed sequence read, or a stitched read. Furthermore, a read segment can refer to an individual nucleotide base, such as a single nucleotide variant.

The term “single nucleotide variant” or “SNV” refers to a substitution of one nucleotide to a different nucleotide at a position (e.g., site) of the nucleotide in a sequence, e.g., a sequence read from an individual. A substitution from a first nucleobase “X” to a second nucleobase “Y” can be denoted as “X>Y.” For example, a cytosine to thymine SNV can be denoted as “C>T.”

The term “indel” refers to any insertion or deletion of one or more bases having a length and a position (which can also be referred to as an anchor position) in a sequence read. An insertion corresponds to a positive length, while a deletion corresponds to a negative length.

The term “mutation” refers to one or more SNVs or indels.

The term “true positive” refers to a SNV or mutation that indicates real biology, for example, presence of a potential cancer, disease, or germline mutation in an individual. True positives are not caused by mutations naturally occurring in healthy individuals (e.g., recurrent mutations) or other sources of artifacts such as process errors during assay preparation of nucleic acid samples.

The term “false positive” refers to a mutation incorrectly determined to be a true positive. Generally, false positives can be more likely to occur when processing sequence reads associated with greater mean noise rates or greater uncertainty in noise rates.

The term “cell free nucleic acid,” “cell free DNA,” or “cfDNA” refers to nucleic acid fragments that circulate in an individual's body (e.g., bloodstream) and originate from one or more healthy cells and/or from one or more cancer cells.

The term “circulating tumor DNA” or “ctDNA” refers to deoxyribonucleic acid fragments that originate from tumor cells or other types of cancer cells, which can be released into an individual's bloodstream, for example, as result of biological processes such as apoptosis or necrosis of dying cells or actively released by viable tumor cells. The term “circulating tumor RNA” or “ctRNA” refers to ribonucleic acid fragments that originate from tumor cells or other types of cancer cells, which can be released into an individual's bloodstream, for example, as result of biological processes such as apoptosis or necrosis of dying cells or actively released by viable tumor cells.

The term “genomic nucleic acid,” “genomic DNA,” or “gDNA” refers to nucleic acid including chromosomal DNA that originate from one or more healthy cells. In various embodiments, gDNA can be extracted from healthy cells (e.g., non-tumor cells) or from tumor cells (e.g., a biopsy sample). In some embodiments, gDNA can be extracted from a cell derived from a blood cell lineage, such as a white blood cell.

The term “DNA fragment,” “fragment,” or “DNA molecule” may generally refer to any deoxyribonucleic acid fragments, i.e., cfDNA, gDNA, ctDNA, etc.

The term “methylation pattern” refers to methylation statuses of CpG sites on a nucleic acid fragment.

The term “anomaly score” refers to a score for a methylation sequence read indicating likelihood of observing such methylation pattern in healthy samples. In various embodiments, the anomaly score is a p-value that represents a calibrated likelihood of observing the methylation pattern given a trained probabilistic noise model corresponding to a genomic region that the fragment overlaps.

The term “anomalous fragment,” “anomalously methylated fragment,” or “fragment with an anomalous methylation pattern” refers to a fragment that has anomalous methylation of CpG sites. Anomalous methylation of a fragment may be determined using probabilistic noise models to identify unexpectedness of observing a fragment's methylation pattern in a control group.

The term “unusual fragment with extreme methylation” or “UFXM” refers to a hypomethylated fragment or a hypermethylated fragment. A hypomethylated fragment and a hypermethylated fragment refers to a fragment with at least some number of CpG sites (e.g., 5) that have over some threshold percentage (e.g., 90%) of methylation or unmethylation, respectively.

The term “alternative allele,” “alternate allele,” or “ALT” refers to an allele having one or more mutations relative to a reference allele, e.g., corresponding to a known gene.

The term “sequencing depth” or “depth” refers to a total number of sequence reads or read segments derived from the same position or location of the genome from a sample obtained from an individual.

The term “alternate depth” or “AD” refers to a number of sequence reads or read segments in a sample that support an ALT, e.g., include mutations of the ALT.

The term “reference depth” refers to a number of sequence reads or read segments in a sample that include a reference allele at a candidate variant location.

The term “alternate frequency,” “allele frequency,” or “AF” refers to the frequency of a given ALT. The AF can be determined by dividing the corresponding AD of a sample by the depth of the sample for the given ALT.

The term “variant” or “true variant” refers to a SNV or mutated nucleotide base at a position in the genome. Such a variant can be indicative of, or may lead to, the development and/or progression of cancer in an individual.

The term “edge variant” refers to a mutation located near an edge of a sequence read, for example, within a threshold distance of nucleotide bases from the edge of the sequence read.

The term “candidate variant,” “called variant,” “putative variant,” or refers to one or more detected nucleotide variants of a nucleotide sequence, for example, at a position in the genome that is determined to be mutated. Generally, a nucleotide base is deemed a called variant based on the presence of an alternative allele on sequence reads obtained from a sample, where the sequence reads each cross over the position in the genome. The source of a candidate variant can initially be unknown or uncertain. During processing, candidate variants can be associated with an expected source such as gDNA (e.g., blood-derived) or cells impacted by cancer (e.g., tumor-derived). Additionally, candidate variants can be called as true positives.

The term “non-edge variant” refers to a candidate variant that is not determined to be resulting from an artifact process, e.g., using an edge variant filtering method described herein. In some scenarios, a non-edge variant may not be a true variant (e.g., mutation in the genome) as the non-edge variant could arise due to a different reason as opposed to one or more artifact processes.

The term “biological sample,” “patient sample,” or “sample” refers to any sample taken from a subject, which can reflect a biological state associated with the subject, and that includes genetic material, e.g., cell-free DNA. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject. A biological sample can include any tissue or material derived from a living or dead subject. A biological sample can be a cell-free sample. A biological sample can comprise a nucleic acid (e.g., DNA or RNA) or a fragment thereof. The term “nucleic acid” can refer to deoxyribonucleic acid (DNA), ribonucleic acid (RNA) or any hybrid or fragment thereof. The nucleic acid in the sample can be a cell-free nucleic acid. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A biological sample can be a stool sample. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis. Once the nucleic acid fragments of a sample are sequenced, an analytics system may electronically represent the sample as comprising the sequence reads.

The terms “control,” “control sample,” “reference,” “reference sample,” “healthy sample,” and “normal sample” describe a sample from a subject that does not have a particular condition, or is otherwise healthy. In an example, a method as disclosed herein can be performed on a subject having a tumor, where the reference sample is a sample taken from a healthy tissue of the subject. A reference sample can be obtained from the subject, or from a database. The reference can be, e.g., a reference genome that is used to map nucleic acid fragment sequences obtained from sequencing a sample from the subject. A reference genome can refer to a haploid or diploid genome to which nucleic acid fragment sequences from the biological sample and a constitutional sample can be aligned and compared. An example of a constitutional sample can be DNA of white blood cells obtained from the subject. For a haploid genome, there can be only one nucleotide at each locus. For a diploid genome, heterozygous loci can be identified; each heterozygous locus can have two alleles, where either allele can allow a match for alignment to the locus.

The term “cancer” or “tumor” refers to an abnormal mass of tissue in which the growth of the mass surpasses and is not coordinated with the growth of normal tissue.

The phrase “healthy,” refers to a subject possessing good health. A healthy subject can demonstrate an absence of any malignant or non-malignant disease. A “healthy individual” can have other diseases or conditions, unrelated to the condition being assayed, which can normally not be considered “healthy.”

The term “methylation” refers to a modification of deoxyribonucleic acid (DNA) where a hydrogen atom on the pyrimidine ring of a cytosine base is converted to a methyl group, forming 5-methylcytosine. In particular, methylation tends to occur at dinucleotides of cytosine and guanine referred to herein as “CpG sites.” In other instances, methylation may occur at a cytosine not part of a CpG site or at another nucleotide that's not cytosine; however, these are rarer occurrences. Anomalous cfDNA methylation can be identified as hypermethylation or hypomethylation, both of which may be indicative of cancer status. DNA methylation anomalies (compared to healthy controls) can cause different effects, which may contribute to cancer. The principles described herein are equally applicable for the detection of methylation in a CpG context and non-CpG context, including non-cytosine methylation. Further, the methylation state vectors may contain elements that are generally vectors of sites where methylation has or has not occurred (even if those sites are not CpG sites specifically).

The term “methylation fragment” or “nucleic acid methylation fragment” refers to a sequence of methylation states for each CpG site in a plurality of CpG sites, determined by a methylation sequencing of nucleic acids (e.g., a nucleic acid molecule and/or a nucleic acid fragment). In a methylation fragment, a location and methylation state for each CpG site in the nucleic acid fragment is determined based on the alignment of the sequence reads (e.g., obtained from sequencing of the nucleic acids) to a reference genome. A nucleic acid methylation fragment comprises a methylation state of each CpG site in a plurality of CpG sites (e.g., a methylation state vector), which specifies the location of the nucleic acid fragment in a reference genome (e.g., as specified by the position of the first CpG site in the nucleic acid fragment using a CpG index, or another similar metric) and the number of CpG sites in the nucleic acid fragment. Alignment of a sequence read to a reference genome, based on a methylation sequencing of a nucleic acid molecule, can be performed using a CpG index. As used herein, the term “CpG index” refers to a list of each CpG site in the plurality of CpG sites (e.g., CpG 1, CpG 2, CpG 3, etc.) in a reference genome, such as a human reference genome, which can be in electronic format. The CpG index further comprises a corresponding genomic location, in the corresponding reference genome, for each respective CpG site in the CpG index. Each CpG site in each respective nucleic acid methylation fragment is thus indexed to a specific location in the respective reference genome, which can be determined using the CpG index.

The terminology used herein is for the purpose of describing particular cases only and is not intended to be limiting. As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

### I.D. Example Analytics System

FIG. 2A is a flowchart of devices for sequencing nucleic acid samples according to one embodiment. This illustrative flowchart includes devices such as a sequencer 220 and an analytics system 200. The sequencer 220 and the analytics system 200 may work in tandem to perform one or more steps in any of the processes described herein this disclosure.

In various embodiments, the sequencer 220 receives an enriched nucleic acid sample 210. As shown in FIG. 2A, the sequencer 220 can include a graphical user interface 225 that enables user interactions with particular tasks (e.g., initiate sequencing or terminate sequencing) as well as one more loading stations 230 for loading a sequencing cartridge including the enriched fragment samples and/or for loading necessary buffers for performing the sequencing assays. Therefore, once a user of the sequencer 220 has provided the necessary reagents and sequencing cartridges to the loading station 230 of the sequencer 220, the user can initiate sequencing by interacting with the graphical user interface 225 of the sequencer 220. Once initiated, the sequencer 220 performs the sequencing and outputs the sequence reads of the enriched fragments from the nucleic acid sample 210.

In some embodiments, the sequencer 220 is communicatively coupled with the analytics system 200. The analytics system 200 includes some number of computing devices used for processing the sequence reads for various applications such as assessing methylation status at one or more CpG sites, variant calling or quality control. The sequencer 220 may provide the sequence reads in a BAM file format to the analytics system 200. The analytics system 200 can be communicatively coupled to the sequencer 220 through a wireless, wired, or a combination of wireless and wired communication technologies. Generally, the analytics system 200 is configured with a processor and non-transitory computer-readable storage medium storing computer instructions that, when executed by the processor, cause the processor to process the sequence reads or to perform one or more steps of any of the methods or processes disclosed herein.

In some embodiments, the sequence reads may be aligned to a reference genome using known methods in the art to determine alignment position information. Alignment position may generally describe a beginning position and an end position of a region in the reference genome that corresponds to a beginning nucleotide based and an end nucleotide base of a given sequence read. Corresponding to methylation sequencing, the alignment position information may be generalized to indicate a first CpG site and a last CpG site included in the sequence read according to the alignment to the reference genome. The alignment position information may further indicate methylation statuses and locations of all CpG sites in a given sequence read. A region in the reference genome may be associated with a gene or a segment of a gene; as such, the analytics system 200 may label a sequence read with one or more genes that align to the sequence read. In one embodiment, fragment length (or size) is determined from the beginning and end positions.

In various embodiments, for example when a paired-end sequencing process is used, a sequence read is comprised of a read pair denoted as R_1 and R_2. For example, the first read R_1 may be sequenced from the first end of a double-stranded DNA (dsDNA) molecule whereas the second read R_2 may be sequenced from the second end of the double-stranded DNA (dsDNA). Therefore, nucleotide base pairs of the first read R_1 and second read R_2 may be aligned consistently (e.g., in opposite orientations) with nucleotide bases of the reference genome. Alignment position information derived from the read pair R_1 and R_2 may include a beginning position in the reference genome that corresponds to an end of a first read (e.g., R_1) and an end position in the reference genome that corresponds to an end of a second read (e.g., R_2). In other words, the beginning position and end position in the reference genome represent the likely location within the reference genome to which the nucleic acid fragment corresponds. An output file having SAM (sequence alignment map) format or BAM (binary) format may be generated and output for further analysis.

FIG. 2B is a block diagram of an analytics system 200 for processing DNA samples according to one embodiment. The analytics system implements one or more computing devices for use in analyzing DNA samples. The analytics system 200 includes a sequence processor 240, a sequence database 245, one or more models 250, a model database 255, a score engine 260, and a parameter database 265. In some embodiments, the analytics system 200 performs some or all of the processes described herein disclosure.

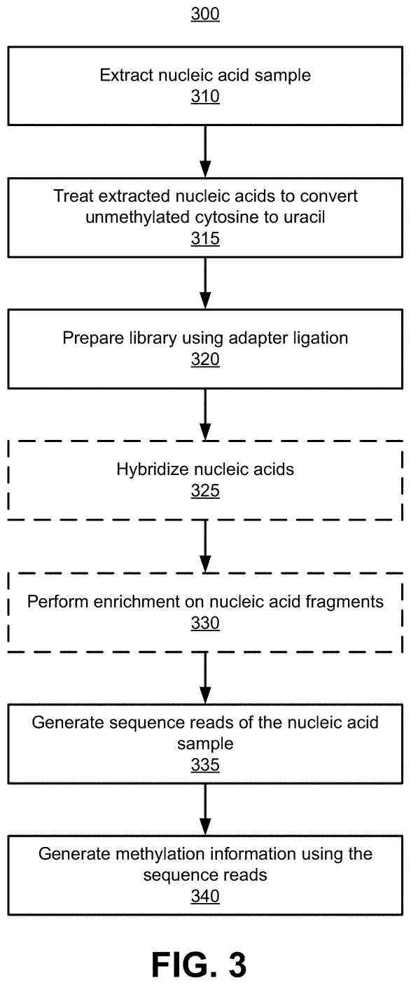

The sequence processor 240 generates methylation state vectors for fragments from a sample. At each CpG site on a fragment, the sequence processor 240 generates a methylation state vector for each fragment specifying a location of the fragment in the reference genome, a number of CpG sites in the fragment, and the methylation state of each CpG site in the fragment whether methylated, unmethylated, or indeterminate via the processes described under FIGS. 3 & 4. The sequence processor 240 may store methylation vectors for fragments in the sequence database 245. Data in the sequence database 245 may be organized such that the methylation state vectors from a sample are associated to one another.

Further, multiple different models 250 may be stored in the model database 255 or retrieved for use with test samples. The models 250 may include the probabilistic noise models trained for the genomic regions of the genome and the trained cancer classifier for determining a cancer prediction for a test sample using a feature vector derived from anomalous fragments. Training of the probabilistic noise models for determining a fragment anomaly score is discussed further in FIG. 5A. Training and use of the cancer classifier is further discussed in FIGS. 6A & 6B. The analytics system 200 may train the one or more models 250 and store various trained parameters in the parameter database 265. The analytics system 200 stores the models 250 along with functions in the model database 255.

During inference, the score engine 260 uses the one or more models 250 to return outputs. The score engine 260 accesses the models 250 in the model database 255 along with trained parameters from the parameter database 265. According to each model, the score engine receives an appropriate input for the model and calculates an output based on the received input, the parameters, and a function of each model relating the input and the output. In some use cases, the score engine 260 further calculates metrics correlating to a confidence in the calculated outputs from the model. In other use cases, the score engine 260 calculates other intermediary values for use in the model.

### II. Example Assay Protocol

FIG. 3 is a flowchart describing a process 300 of sequencing nucleic acids, according to an embodiment. In some embodiments, the process 300 is performed to generate the methylation information (measure of methylated CpG sites) used in the cancer classification workflow 100. The process 300 of sequencing nucleic acids may be performed by the sequencer 220 and the analytics system 200 working in conjunction.

In step 310, a nucleic acid sample (e.g., DNA or RNA) is extracted from a subject. In the present disclosure, DNA and RNA can be used interchangeably unless otherwise indicated. That is, the embodiments described herein can be applicable to both DNA and RNA types of nucleic acid sequences. However, the examples described herein can focus on DNA for purposes of clarity and explanation. The sample can include nucleic acid molecules derived from any subset of the human genome, including the whole genome. The sample can include blood, plasma, serum, urine, fecal, saliva, other types of bodily fluids, or any combination thereof. In some embodiments, methods for drawing a blood sample (e.g., syringe or finger prick) can be less invasive than procedures for obtaining a tissue biopsy, which can require surgery. The extracted sample can comprise cfDNA and/or ctDNA. If a subject has a disease state, such as cancer, cell free nucleic acids (e.g., cfDNA) in an extracted sample from the subject generally includes detectable level of the nucleic acids that can be used to assess a disease state.

In step 315, the extracted nucleic acids (e.g., including cfDNA fragments) are treated to convert unmethylated cytosines to uracils. In some embodiments, the method 300 uses a bisulfite treatment of the samples which converts the unmethylated cytosines to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, Calif.)) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, e.g., APOBEC-Seq (NEBiolabs, Ipswich, Mass.).

In step 320, a sequencing library is prepared. In some embodiments, the preparation includes at least two steps. In a first step, a ssDNA adapter is added to the 3′-OH end of a bisulfite-converted ssDNA molecule using a ssDNA ligation reaction. In some embodiments, the ssDNA ligation reaction uses CircLigase II (Epicentre) to ligate the ssDNA adapter to the 3′-OH end of a bisulfite-converted ssDNA molecule, wherein the 5′-end of the adapter is phosphorylated and the bisulfite-converted ssDNA has been dephosphorylated (i.e., the 3′ end has a hydroxyl group). In another embodiment, the ssDNA ligation reaction uses Thermostable 5′ AppDNA/RNA ligase (available from New England BioLabs (Ipswich, Mass.)) to ligate the ssDNA adapter to the 3′-OH end of a bisulfite-converted ssDNA molecule. In this example, the first UMI adapter is adenylated at the 5′-end and blocked at the 3′-end. In another embodiment, the ssDNA ligation reaction uses a T4 RNA ligase (available from New England BioLabs) to ligate the ssDNA adapter to the 3′-OH end of a bisulfite-converted ssDNA molecule.

In a second step, a second strand DNA is synthesized in an extension reaction. For example, an extension primer, that hybridizes to a primer sequence included in the ssDNA adapter, is used in a primer extension reaction to form a double-stranded bisulfite-converted DNA molecule. Optionally, in some embodiments, the extension reaction uses an enzyme that is able to read through uracil residues in the bisulfite-converted template strand.

Optionally, in a third step, a dsDNA adapter is added to the double-stranded bisulfite-converted DNA molecule. Then, the double-stranded bisulfite-converted DNA can be amplified to add sequencing adapters. For example, PCR amplification using a forward primer that includes a P5 sequence and a reverse primer that includes a P7 sequence is used to add P5 and P7 sequences to the bisulfite-converted DNA. Optionally, during library preparation, unique molecular identifiers (UMI) can be added to the nucleic acid molecules (e.g., DNA molecules) through adapter ligation. The UMIs are short nucleic acid sequences (e.g., 4-10 base pairs) that are added to ends of DNA fragments during adapter ligation. In some embodiments, UMIs are degenerate base pairs that serve as a unique tag that can be used to identify sequence reads originating from a specific DNA fragment. During PCR amplification following adapter ligation, the UMIs are replicated along with the attached DNA fragment, which provides a way to identify sequence reads that came from the same original fragment in downstream analysis.

In an optional step 325, the nucleic acids (e.g., fragments) can be hybridized. Hybridization probes (also referred to herein as “probes”) can be used to target, and pull down, nucleic acid fragments informative for disease states. For a given workflow, the probes can be designed to anneal (or hybridize) to a target (complementary) strand of DNA or RNA. The target strand can be the “positive” strand (e.g., the strand transcribed into mRNA, and subsequently translated into a protein) or the complementary “negative” strand. The probes can range in length from 10s, 100s, or 1000s of base pairs. Moreover, the probes can cover overlapping portions of a target region.

In an optional step 330, the hybridized nucleic acid fragments are captured and can be enriched, e.g., amplified using PCR. In some embodiments, targeted DNA sequences can be enriched from the library. This is used, for example, where a targeted panel assay is being performed on the samples. For example, the target sequences can be enriched to obtain enriched sequences that can be subsequently sequenced. In general, any known method in the art can be used to isolate, and enrich for, probe-hybridized target nucleic acids. For example, as is well known in the art, a biotin moiety can be added to the 5′-end of the probes (i.e., biotinylated) to facilitate isolation of target nucleic acids hybridized to probes using a streptavidin-coated surface (e.g., streptavidin-coated beads).

In some embodiments, one or more (or all) of the probes are designed based on a gene panel to analyze particular mutations or target regions of the genome (e.g., of the human or another organism) that are suspected to correspond to certain cancers or other types of diseases. By using a targeted gene panel rather than sequencing all expressed genes of a genome, also known as “whole exome sequencing,” the method 300 can be used to increase sequencing depth of the target regions, where depth refers to the count of the number of times a given target sequence within the sample has been sequenced. Increasing sequencing depth reduces required input amounts of the nucleic acid sample.

In step 335, sequence reads are generated from the nucleic acid sample, e.g., enriched sequences. Sequencing data can be acquired from the enriched DNA sequences by known means in the art. For example, the method can include next generation sequencing (NGS) techniques including synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators.

In step 340, the sequence processor 210 can generate methylation information using the sequence reads. In one or more embodiments, methylation information includes a methylation vector comprising methylation statuses for CpG sites on the nucleic acid fragment.

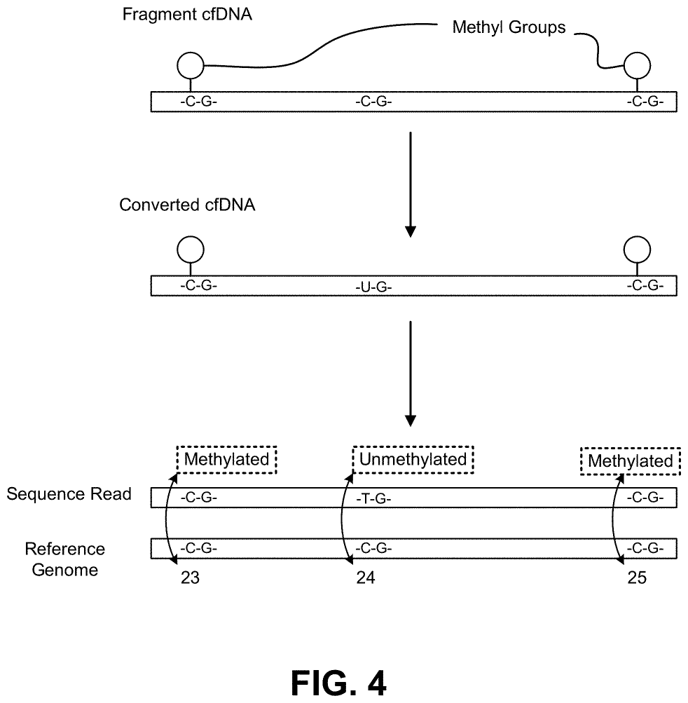

FIG. 4 is an illustration of a part of the process of FIG. 3 of sequencing nucleic acids to obtain a methylation information, according to an embodiment. As an example, a cfDNA fragment includes three CpG sites. As shown by the methyl groups, the first and third CpG sites of the cfDNA fragment are methylated. During the treatment step 315, the cfDNA fragment is converted to generate a converted cfDNA fragment. During the treatment, the second CpG site that was unmethylated has a cytosine converted to uracil. However, the first and third CpG sites are not converted.

After the treatment, a sequencing library is prepared and the sequence processor 210 generates sequence reads. In an embodiment, the sequence processor 210 aligns a sequence read to a reference genome. The reference genome provides the context as to a position in a human genome from which the cfDNA fragment originates. The sequence processor 210 aligns the sequence read such that the three CpG sites correlate to CpG sites 23, 24, and 25 (arbitrary reference identifiers used for convenience of description). The sequence processor 210 thus can generate information both on methylation status of all CpG sites on the cfDNA fragment and which to position in the human genome the CpG sites map. As shown, the CpG sites on the sequence read that were methylated are read as cytosines. A methylation vector may collate the methylation states for each of the CpG sites covered by a fragment.

In this example, the cytosine bases appear in the sequence read only in the first and third CpG site, which allows the sequence processor 210 to infer that the first and third CpG sites in the original cfDNA fragment were methylated. Additionally, the second CpG site is read as a thymine (U is converted to T during the sequencing process), and thus, the sequence processor 210 can infer that the second CpG site was unmethylated in the original cfDNA fragment. With the methylation status and location, the sequence processor 210 generates methylation information (e.g., for determining mean and dispersion of a measure of methylated CpG sites in a region) for the cfDNA fragment. In some embodiments, the methylation information is represented by a methylation vector <M₂₃, U₂₄, M₂₅>, wherein M corresponds to a methylated CpG site, U corresponds to an unmethylated CpG site, and the subscript number corresponds to a position of each CpG site in the reference genome.

### III. Methylation Fragment Probabilistic Noise Model

FIG. 5A is a flowchart of a method 500 for training one or more probabilistic noise models. The analytics systems trains 505 a probabilistic noise model for each region of a set of regions of a genome. The probabilistic noise model may be parameterized by a mean and a dispersion of a measure of methylated CpG sites in a first set (e.g., training data) of nucleic acid fragments from healthy samples.

The analytics system obtains 510 methylation sequence reads from the healthy samples. In various embodiments, the measure of methylated CpG sites can be obtained using the method 300 of FIG. 3. The nucleic acid fragments from a sample can include cfDNA shed from a mixture of species from different tissues or from tissue biopsies. The healthy samples are, generally, without any pre-existing conditions or without any cancer or other disease diagnosis.

The analytics system trains 520 a probabilistic noise model for a genomic region that is parametrized by a mean and a dispersion of methylated CpG sites based on the methylation sequence reads overlapping the genomic region. There can be hundreds, thousands, or more regions of the genome. In some embodiments, there are at least 1,000, at least 2,000, at least 3,000, at least 4,000, at least 5,000, at least 6,000, at least 7,000, at least 8,000, at least 9,000, at least 10,000 genomic regions, at least 20,000 genomic regions, at least 30,000 genomic region, at least 40,000 genomic regions, at least 50,000 genomic regions, at least 60,000 genomic regions, at least 70,000 genomic regions, at least 80,000 genomic regions, or at least 100,000 genomic regions. In some embodiments, each genomic region is no greater than 50, no greater than 60, no greater than 70, no greater than 80, no greater than 90, or no greater than 100 CpG sites. In some such embodiments, each genomic region in the plurality of regions comprises at least 1, at least 2, at least 3, at least 4, at least 5, at least 6, at least 7, at least 8, at least 9, at least 10, at least 20, at least 30, or more than 30 CpG sites. In some embodiments, each genomic region comprises one or more adjacent CpG sites. Genomic regions can be selected based on the proximity of CpG sites within a genomic region. For example, genomic regions are selected based on a threshold density of CpG sites within a genomic region of a predetermined length. The analytics system may segregate the methylation sequence reads based on which of the genomic regions each methylation sequence read overlaps. In other words, the analytics system may, for each genomic region, aggregate methylation sequence reads overlapping the genomic region.

The probabilistic noise model can provide a baseline (e.g., noise) level of CpG sites per region based on the non-cancer samples used for training. In various embodiments, for a given genomic region, the number of methylated CpG sites of a fragment y is modeled using a beta-binomial random variable with a mean parameter φ and a dispersion parameter κ, where N represents the number of CpG sites in the fragment:

y˜beta_binomial(N,φ·κ,(1−φ)·κ)

The mean parameter φ represents an average level of methylated CpG sites in the training data and the dispersion parameter κ represents a variability of methylated CpG sites among tissue types. Values of the mean and dispersion parameters can vary between different regions. Training the probabilistic noise model can include determining posterior distributions of the mean and dispersion parameters for each of the genomic regions using Bayesian inference.

In another implementation, the beta-binomial function can be defined as:

$$f ( n , α , β ) = ( n k ) \frac{B ( k + α , n - k + β )}{B ( α , β )}$$

The likelihood of observing fragment k is defined by the parametrization of the beta-binomial distribution parametrized by n referring to the non-cancer samples and α and β are parameters adjusted to fit the observed non-cancer training samples. The Bayesian inference can be determined using Markov chain Monte Carlo or other suitable algorithms.

Each trained probabilistic noise model can be configured to input a methylation vector for a nucleic acid fragment and to output an anomaly score for the nucleic acid fragment. The anomaly score can indicate a likelihood of observing a fragment having that methylation vector from a population of non-cancer samples. In various embodiments, the anomaly score is a p-value that represents a calibrated likelihood of observing a fragment given the trained probabilistic noise model. In other words, the p-value can indicate a probability that the nucleic acid fragment from the test samples is anomalously methylated. A smaller p-value can correspond to a lower likelihood of observing a fragment and thus may indicate a greater likelihood of anomalous methylation or a disease state. In some embodiments, the analytics system applies a transformation to the p-value, for example, by applying a logarithmic or nonlinear function.

FIG. 5B is a flowchart of a method 530 for utilizing the trained probabilistic noise models, according to some embodiments. The analytics system may train the probabilistic noise models according to the method 500 shown and described in FIG. 5A. The analytics system may store the parameters defining the probabilistic noise models in the model database 255 of FIG. 2B.

The analytics system obtains 540 a methylation sequence read for a sample. The sample may be a training sample or a test sample. The methylation sequence read comprises at least methylation statuses for one or more CpG sites in the genome.

The analytics system identifies 550 a genomic region that the methylation sequence read overlaps. The analytics system may identify the genomic region based on the CpG sites that the methylation sequence read overlaps. For example, a first genomic region may cover a series of CpG sites on one chromosome. The analytics system identifies the methylation sequence read as overlapping the same series of CpG sites on the one chromosome, thus overlapping the genomic region.

The analytic system applies 560 the trained probabilistic noise model for the identified genomic region to the methylation sequence read to determine an anomaly score. The analytics system may input the methylation vector (e.g., as determined via method 300 of FIG. 3) into the trained probabilistic noise model which outputs an anomaly score for the methylation vector.

FIG. 6A is a flowchart of a method 600 for training a classifier to determine a cancer prediction from nucleic acid fragments of a sample, according to some embodiments. The method 600 may be performed by an analytics system, an example of which is provided in FIGS. 2A & 2B. The analytics system is capable of sequencing samples comprised of nucleic acid fragments and performing various analyses on the sequence reads of the nucleic acid fragments. The analytics system may train various models to perform the analyses, including a classifier that is capable of detecting a presence of a cancer state, a stage of cancer, a tumor fraction, another disease state, or some combination thereof. In one or more other embodiments, the method 600 may include additional steps, fewer steps, steps in a different order, or some combination thereof.

In some embodiments, the method 600 includes obtaining a sample from an individual. The sample can include cell free nucleic acid. In addition, the sample can include blood, plasma, serum, urine, fecal, saliva, other types of bodily fluids, or any combination thereof. The analytics system generates a set of sequence reads using the sample. According to the present disclosure, the analytics system generates a methylation vector for each fragment that is sequenced. Methylation can occur at CpG sites throughout the human genome. A CpG site is a location in a region of a genome including a guanine (G) nucleotide after a cytosine (C) nucleotide. In a methylated CpG site, the cytosine is methylated such that a methyl group is added to the nucleic acid molecule. Certain regions in the human genome can have a frequency of methylated CpG sites greater than that of other regions. Methylation states of CpG sites in a region can have similar characteristics due to locally coordinated activities of methylation enzymes. Example discussion relating to methylation sequencing is described in FIGS. 3 and 4.

The analytics system, for each training sample, determines 610 an anomaly score for each fragment using the trained probabilistic noise models. The analytics system can input each methylation vector for each fragment into the appropriate probabilistic noise model. For example, a first fragment overlaps a first region of the plurality of regions. A first probabilistic noise model can be trained for the first region. The analytics system can input the methylation vector of the first fragment into the first probabilistic noise model to generate an anomaly score for the first fragment. The training samples may include a non-cancer cohort of non-cancer samples and one or more cohorts of cancer samples. Each cohort of cancer samples may be of one cancer type. For example, there is a first cohort of breast cancer samples and a second cohort of lung cancer samples. In one or more embodiments, there is a cohort of White Blood Cell (WBC) samples composed of nucleic acid fragments shed from White Blood Cell tissue, i.e., relating to one or more hematological conditions.

Using the trained probabilistic noise model as a baseline of methylation in healthy samples, the analytics system can detect anomalously methylated fragments that deviate from the baseline. The analytics system, for each training sample, determines 615 a count of anomalously methylated fragments in each region of the plurality of regions by comparing the anomaly scores with a threshold anomaly score. The threshold anomaly score can be a Phred quality score, for example, Q20, Q30, or another threshold. A Q30 threshold can represent a probability of an incorrect base call in 1/1000 base pairs of sequence reads. In some embodiments, with the anomaly score as a p-value, the threshold p-value may be set to 0.0001, 0.001, 0.005, 0.01, 0.02, 0.03, 0.04, 0.05, 0.06, 0.07, 0.08, 0.09, 0.1, or any other value between 0 and 0.5. In one or more embodiments, the analytics system may employ an optimization algorithm to identify the optimal threshold score to use for each region. The analytics system sweeps through the range of candidate threshold scores while analyzing performance on cancer classification (or another suitable metric) and performs a grid search to identify the optimal score based on the performance.

The analytics system, for each training sample, determines 620 a ratio of the count of anomalously methylated fragments in the genomic region to a total number of fragments in the genomic region. As a result, each training sample can have a ratio per region indicating the number of anomalously methylated fragments to total fragments in the genomic region. In other embodiments, the count of anomalously methylated fragments may be normalized in other manners, e.g., based on sequencing depth over all regions.

In one or more embodiments, the analytics system filters 625 one or more regions having an above threshold percentage of WBC samples with at least one anomalously methylated fragment overlapping the one or more regions. A noisy region can be deemed to have an above threshold percentage of WBC samples with at least one anomalously methylated fragment overlapping the genomic region. For example, the threshold percentage may be 5%, 10%, 15%, 20%, 25%, 30%, 35%, and 40%. In other embodiments, the analytics system looks to determine whether there are more than some threshold number or some threshold ratio of anomalously methylated fragments overlapping the genomic region to label the genomic region as a noisy region. For example, at least two, three, four, five, six, seven, eight, nine, or ten anomalously methylated fragments. Or a ratio of at least 1:1000, 1:100, 1:10, etc. The parameters for determining a region to be noisy can be tuned based subsequent training and validation of the classifier. In other embodiments, the analytics system may filter regions using other cohorts of training samples. For example, instead of WBC samples, the analytics system may filter regions having an above threshold percentage of breast cancer samples having at least one anomalously methylated fragment overlapping the genomic regions. The genomic regions that are not filtered can be used in the classification process.

In one or more embodiments, the analytics system may assign weights to each region based on the criteria for filtration described above. In embodiments where there is a binary cutoff used, e.g., more than 20% of WBC samples with at least one anomalously methylated fragment overlapping the genomic region, the weight can be a set value (e.g., 0.5) for regions that surpass the threshold and a default value (e.g., 1) for regions that fall below the threshold. In additional embodiments, the analytics system may utilize a gradation for assigning weights to the genomic regions. Regions that have more than 40% of WBC samples with at least one anomalously methylated fragment overlapping the genomic regions can be assigned a weight of 0. Regions that have between 30% and 40% of WBC samples with at least one anomalously methylated fragment can be assigned a weight of 0.2. Regions that have between 20% and 30% of WBC samples with at least one anomalously methylated fragment can be assigned a weight of 0.5. Regions that have between 10% and 20% of WBC samples with at least one anomalously methylated fragment can be assigned a weight of 0.8. And regions that have below a 10% of WBC samples with at least one anomalously methylated fragment can be assigned a default weight of 1. In other embodiments, the weights can be adaptively adjusted based on performance of a downstream classifier. The analytics system can adjust the ranges based on the performance, e.g., regions assigned the weight of 0.5 are shifted to the range between 15% and 25% to further reduce the influence of regions with lower noise. In effect, regions with sample percentages between 15% and 20% were originally weighted as 0.8, but were reduced to 0.5, thus lowering their influence in the cancer classification.

The analytics system trains 630 a classifier to detect a cancer prediction using the training samples, wherein the ratios determined at step 620 serve as features to the classifier. The analytics system generates a feature vector for each training sample comprising the ratios determined per region in step 620. In embodiments with filtration at step 625, the genomic regions used for featurization exclude the filtered regions. In other embodiments with weights assigned to the genomic regions, the ratios are multiplied by the weights. For example, given that a first region has an assigned weight of 0.3, the feature of a training sample for that first region would be the ratio, e.g., 0.15*0.3=0.045. Each training sample may have a label relaying a cancer status of the sample, e.g., non-cancer, head/neck cancer, prostate cancer, thyroid cancer, or leukemia (as some examples). The classifier can be trained to distinguish the labels of the training samples based on the feature vector generated for the training samples. In one or more embodiments, the classifier can be a machine learning model.

Machine learning may refer to a series of analytic methods and algorithms that can learn from and make predictions on data by building a model. Machine learning is classified as a branch of artificial intelligence that focuses on the development of computer programs that can automatically update and learn to produce predictions when exposed to data. In some embodiments, machine learning is one tool used to create the digital network and personal digital records linking sensed or recorded data with a specific output such as response to therapy, or ability to maintain normal rhythm. For applications in the brain, outputs could include absence of seizure activity. Machine learning techniques include supervised learning, transfer learning, semi-supervised learning, unsupervised learning, or reinforcement learning. Several other classifications may exist. Supervised machine learning may include methods of training of models with training data that are associated with labels. Techniques in supervised machine learning may include methods that can classify a series of related or seemingly unrelated inputs into one or more output classes. Output labels are typically used to train the learning models to the desired output, such as favorable patient outcomes, accurate therapy delivery sites and so on. Supervised learning may also include a technique known as ‘transfer learning’, where a pretrained machine learned model trained on one set of input or task, is retrained or fine-tuned to predict outcomes on another input or task.

In some embodiments, the classifier may implement one or more neural networks. Neural networks may refer to a class of machine learning models that include interconnected nodes that can be used to recognize patterns. Neural networks can be deep or shallow neural networks, convolutional neural networks, recurrent neural networks (gated recurrent units, GRUs, or long short term memory, LSTM, networks), generative adversarial networks, and auto-encoders neural networks. Artificial neural networks can be combined with heuristics, deterministic rules and detailed databases.

Additional details relating to training of the classifier may be found in: U.S. application Ser. No. 16/352,602, filed on Mar. 13, 2019; U.S. application Ser. No. 16/723,716, filed on Dec. 20, 2019; U.S. application Ser. No. 16/723,411, filed on Dec. 20, 2019; and U.S. application Ser. No. 15/931,022, filed on May 13, 2020, all of which are incorporated by reference in their entirety.

The cancer prediction output by the classifier may include a binary prediction between cancer and non-cancer, a multiclass prediction between a plurality of cancer types, a tumor fraction, a stage of cancer, another disease state, or some combination thereof. The disease state can be one of breast cancer, uterine cancer, cervical cancer, ovarian cancer, bladder cancer, urothelial cancer of renal pelvis, renal cancer other than urothelial, prostate cancer, anorectal cancer, colorectal cancer, esophageal cancer, gastric cancer, hepatobiliary cancer arising from hepatocytes, hepatobiliary cancer arising from cells other than hepatocytes, pancreatic cancer, squamous cell cancer of the upper gastrointestinal tract, upper gastrointestinal cancer other than squamous, head and neck cancer, lung cancer, lung adenocarcinoma, small cell lung cancer, squamous cell lung cancer and cancer other than adenocarcinoma or small cell lung cancer, neuroendocrine cancer, melanoma, thyroid cancer, sarcoma, multiple myeloma, lymphoma, and leukemia, and other hematological conditions.

FIG. 6B is a flowchart of a method 640 for determining a cancer prediction for a test sample, according to some embodiments. The method 640 is performed with a test sample composed of nucleic acid fragments. The test sample may an unknown cancer status. The analytics system performs a similar processing (as done on the training samples under the method 640) on the test sample to achieve a methylation vector for each nucleic acid fragment present in the test sample. In one or more other embodiments, the method 640 may include additional steps, fewer steps, steps in a different order, or some combination thereof.

The analytics system determines 650 an anomaly score for each fragment from a test sample using the trained probabilistic noise models. As noted above, a probabilistic noise model can input a methylation vector for a fragment that overlaps the genomic region for which the probabilistic noise model is trained. The probabilistic noise model can output an anomaly score indicating a likelihood of observing the methylation vector in a non-cancer healthy population.

The analytics system determines 655 a count of anomalously methylated fragments in each region used for classification by comparing the anomaly scores of the fragments with the threshold anomaly score (e.g., used in step 615 of the method 600). As noted above in step 625, there may be one or more regions filtered or excluded from classification due to the determination that those regions are noisy regions. In other embodiments, the genomic regions used for classification may be assigned weights based on various criteria discussed above.

The analytics system determines 660 a ratio in each region used for classification of the count of anomalously methylated fragments in the genomic region to a total number of fragments in the genomic region. Other featurization metrics may be used in substitution of the ratio, e.g., a total count of anomalously methylated fragments, a binary count of whether an anomalously methylated fragment overlaps the genomic region, a normalized count, etc. The ratio (or the other featurization metrics) serves as a feature for the test sample. The analytics system may generate a feature vector for the test sample based on the ratios (or the other featurization metrics) which includes a value for each region used for classification. In embodiments with weighted regions, the analytics system may further multiply each featurization metric by the weight for each respective region.

The analytics system determines 665 a cancer prediction using the trained classifier. The analytics system inputs the feature vector for the test sample into the trained classifier which outputs a cancer prediction. As described above, the cancer prediction may be a binary prediction and/or a multiclass prediction. The analytics system may return the cancer prediction to a healthcare provider to provide subsequent treatment options informed by the cancer prediction. In other embodiments, method 640 may be utilized for monitoring of cancer progression within a patient known to have cancer. The method 640 may be used to detect success or failure of a treatment plan, e.g., if cancer signal remains substantially the same or is increasing, then the treatment plan is unsuccessful, and, conversely, if the cancer signal decreases, then the treatment plan is successful.

### IV. Example Results

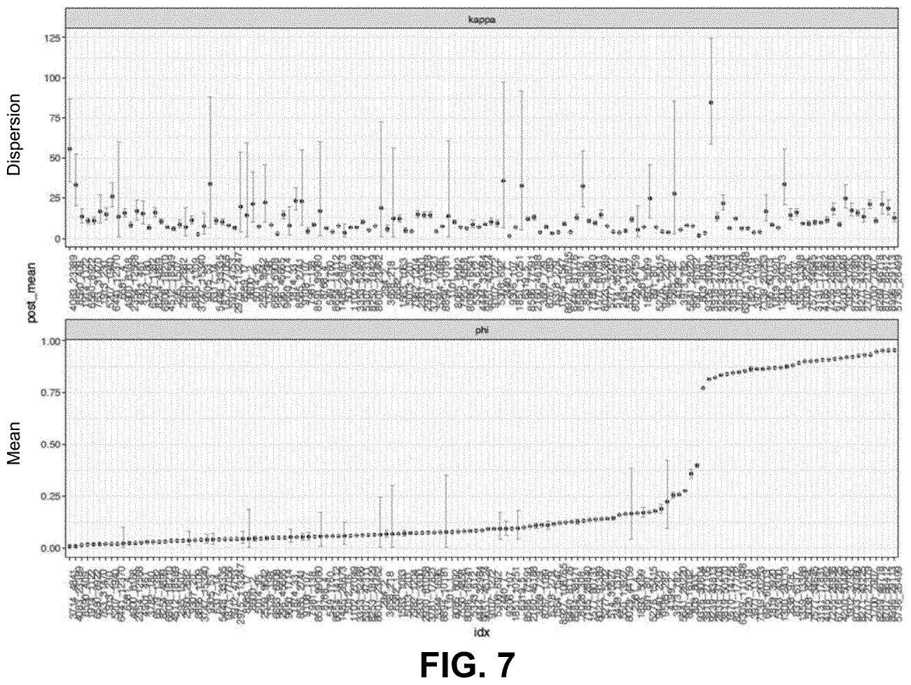

FIG. 7 illustrates posterior distributions of parameters of a probabilistic noise model 230, according to example implementations. The posterior distributions of dispersion and mean parameters of the probabilistic noise model 230 were determined using healthy (non-cancer) training samples. As shown in FIG. 7, the baseline dispersion varies based on the genomic region of the genome. The baseline mean exhibits a bimodal pattern of hypomethylated and hypermethylated regions. In particular, the mean methylation levels of hypomethylated regions are lower than those of the hypermethylated regions. In some embodiments, a hypomethylated region is associated with 10% or less methylated CpG sites, and a hypermethylated region is associated with 90% or more methylated CpG sites. In other embodiments, the threshold percentages for hypermethylated or hypomethylated regions may vary.

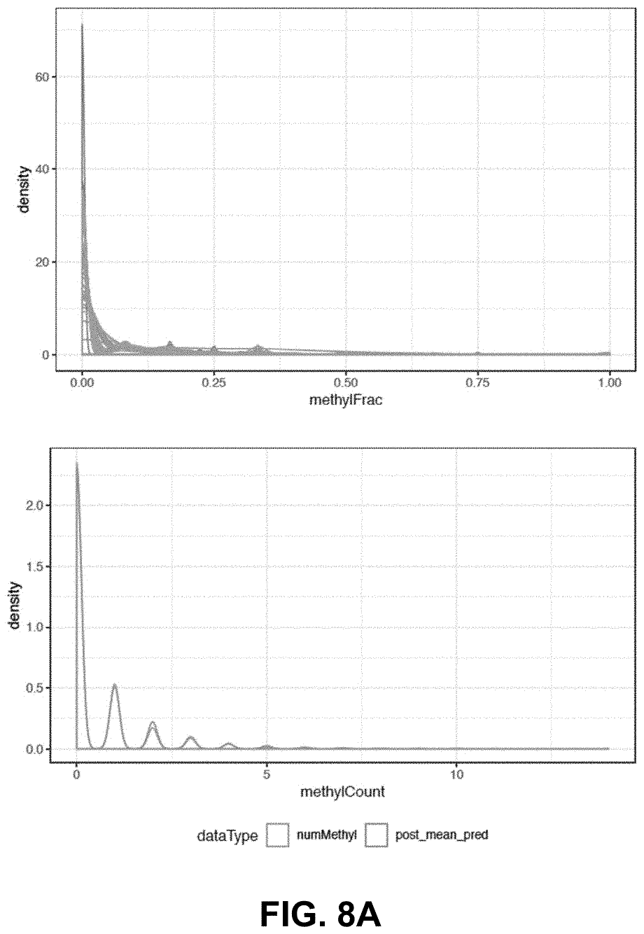

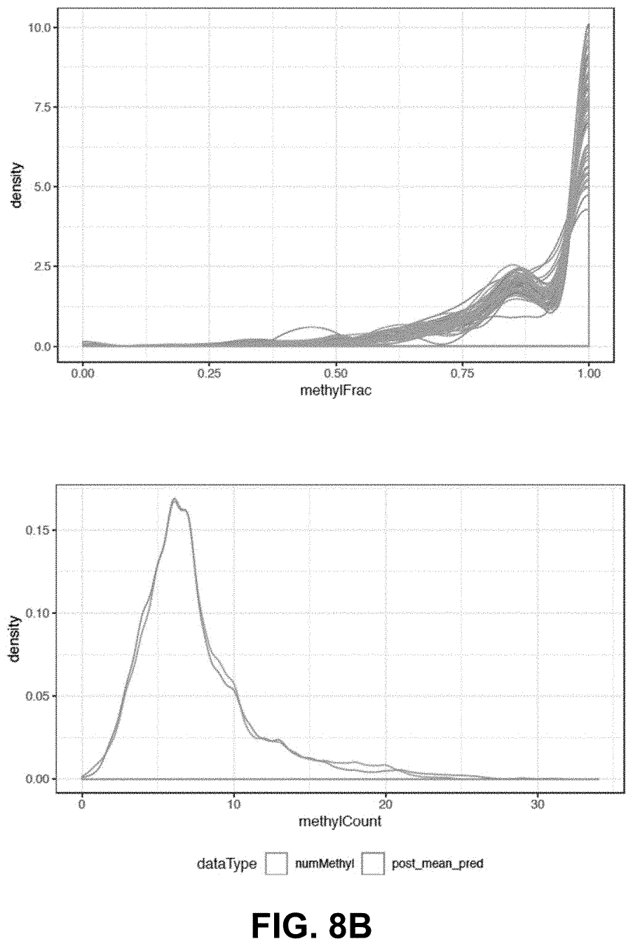

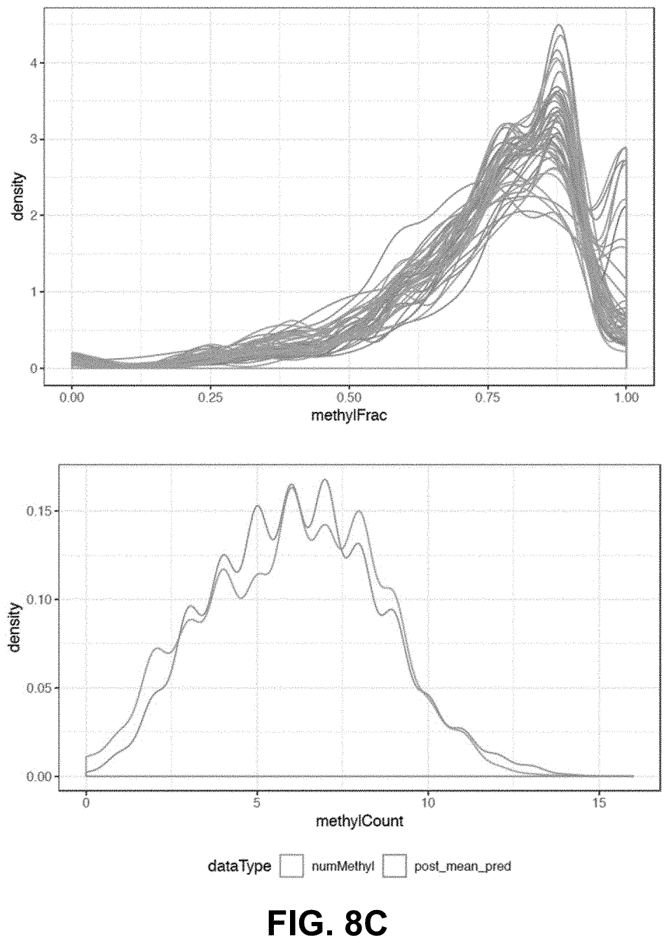

FIGS. 8A, 8B, and 8C illustrate fractions of fragment methylation and counts of methylated CpG sites, according to example implementations. In each of FIGS. 8A, 8B, 8C, the upper graph shows distribution of methylation fractions of real data from training samples, where each curve is associated with a different sample. Each of the lower graphs shows a modeled posterior predictive distribution of methylated CpG counts from holdout test samples, overlaid on the actual distribution (empirical data) from test samples. FIG. 8A includes data from hypomethylated regions and shows that the model generally fits the empirical data. FIG. 8B regions with hypermethylation. FIG. 8C include data from intermediate regions having between 10% and 90% methylated CpG sites.

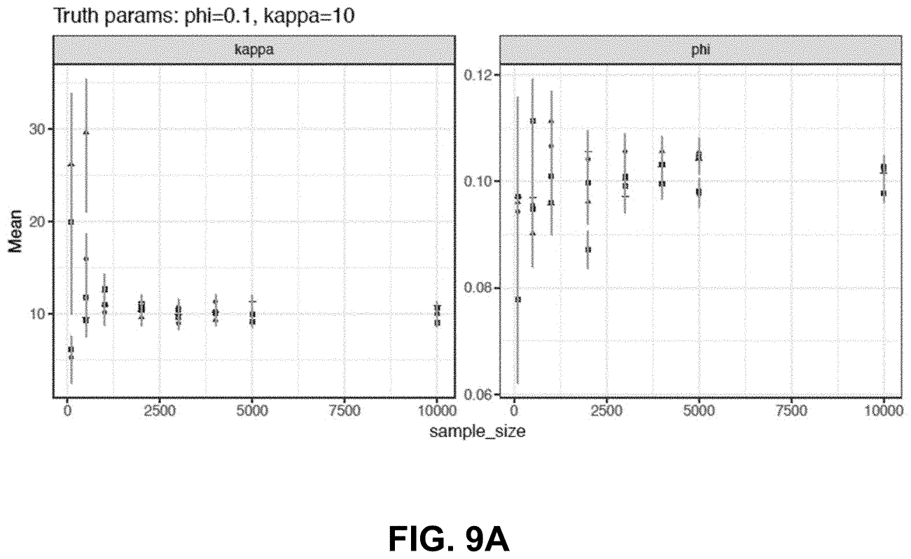

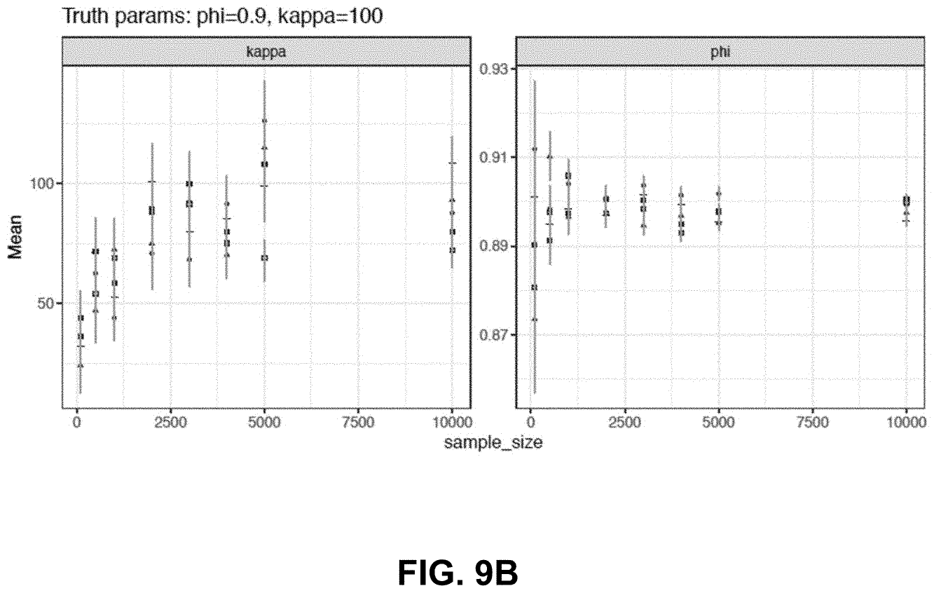

FIGS. 9A and 9B illustrate mean and dispersion parameter estimation using simulations of varying sample size, according to example implementations. FIG. 9A shows parameters for hypomethylated regions, and FIG. 9B shows parameters for hypermethylated regions. In general, as the sample size increases (e.g., up to 5000 fragments), the confidence level of the mean and dispersion parameters improves.

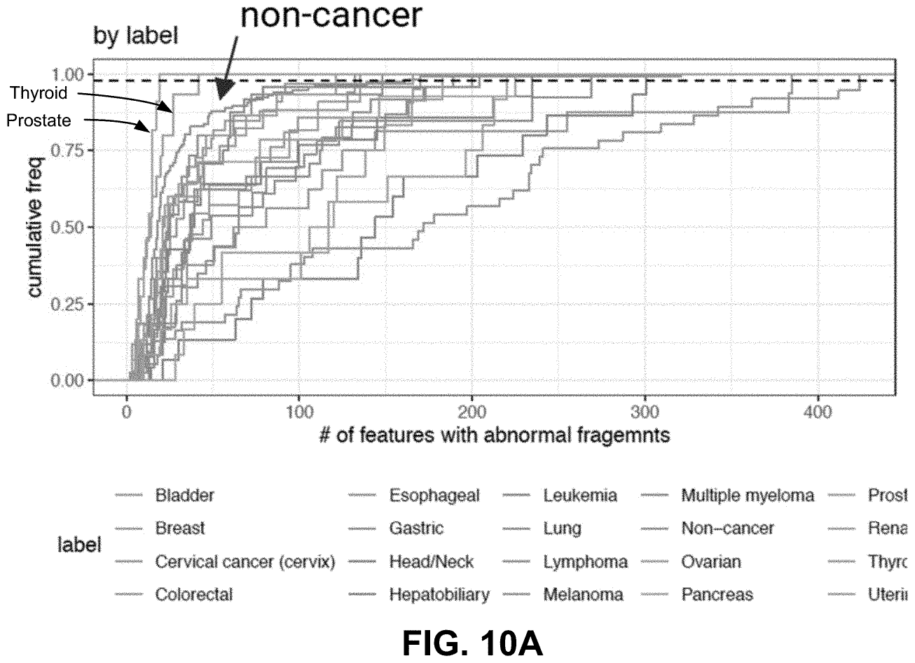

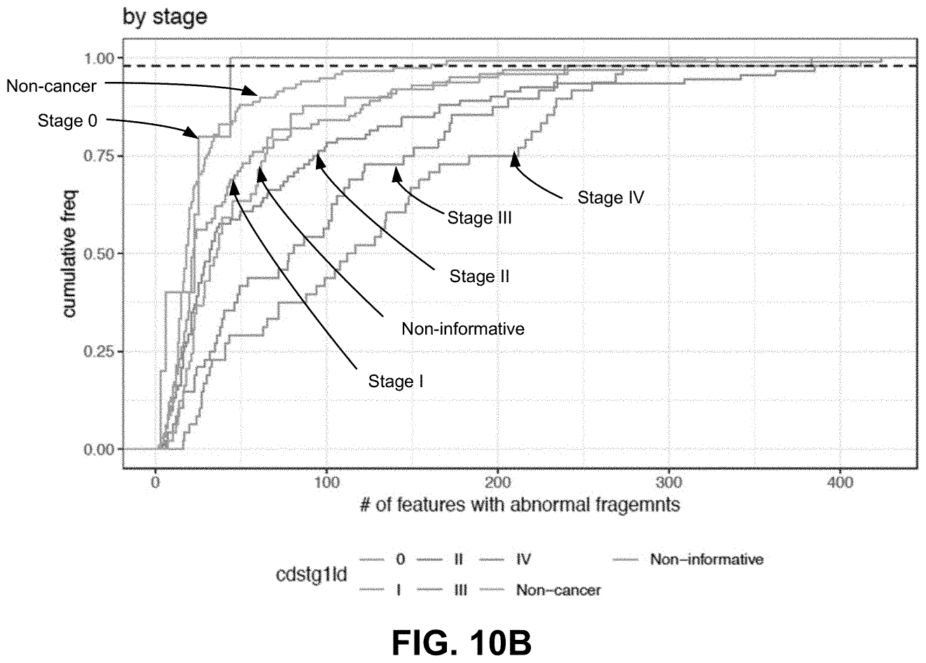

FIG. 10A illustrates cumulative frequency of anomalously methylated fragments by disease state, according to example implementations. The y-axis cumulative frequency represents an additive total probability of a sample, that is, a proportion of the sample that includes at most a number of features with anomalously methylated fragments according to the x-axis. As shown in FIG. 10A, the curves associated with various types of disease states (cancers) can be differentiated from the curve associated with non-cancer. As a result, a trained classifier can predict likelihood of presence of a disease state based on the data illustrated by the separation in the curves. As two outliers, the curves associated with thyroid and prostate cancer have lower tumor fraction because these tissues may tend to shed fewer fragments into blood.

FIG. 10B illustrates cumulative frequency of anomalously methylated fragments by cancer stage, according to example implementations. As the cancer stage progresses from stage 0 up to stage IV, tumor tissues shed greater numbers of fragments, which is concordant with cancer biology. Thus, as shown by the curves in FIG. 10B, the number of features with anomalously methylated fragments increases as the cancer stage progresses. For example, at least 75% of stage I cancer samples have at least approximately 50 anomalously methylated fragments, while at least 75% of stage IV cancer samples have at least 200 anomalously methylated fragments. A trained classifier can predict cancer stage based on the data illustrated by the separation in the curves.

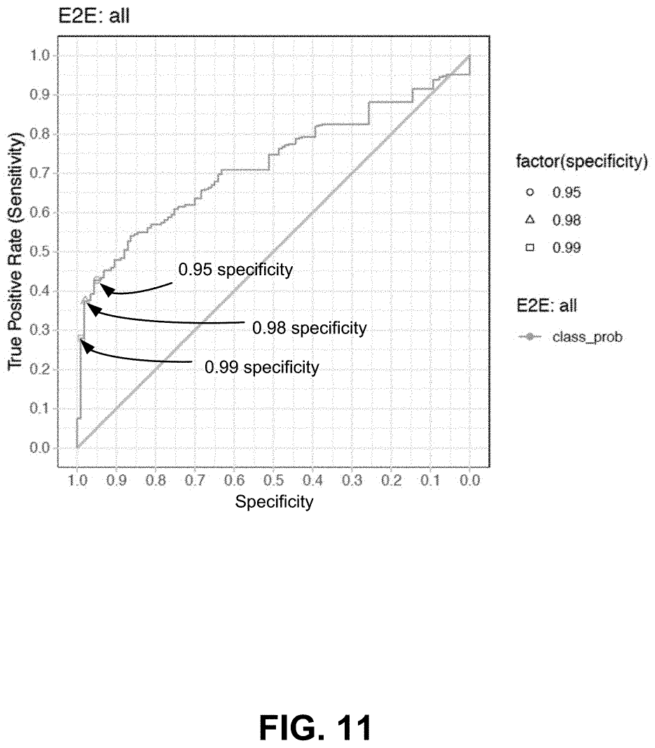

FIG. 11 illustrates a receiver operating characteristic (ROC) curve indicating performance of a trained classifier for detecting anomalously methylated fragments, according to example implementations. As shown in FIG. 11, at 95%, 98%, and 99% specificity, the corresponding sensitivity is between 25% and 45%, and the false positive rate is less than 10%.

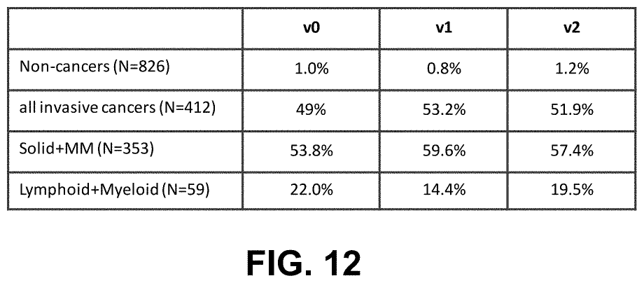

FIG. 12 illustrates a table of the detection rates of the trained classifiers, some classifiers trained with filtered noisy regions, according to example implementations. The table illustrates the comparative performance of trained classifiers that have filtered some regions from classification due to being noisy regions, according to some example implementations. The various classifiers were trained to target a 99.4% Specificity level. The trained classifier labeled “v0” in FIG. 12 serves as a baseline performance, including 20,000 regions in the classification process.

The classifier labeled “v1” used the WBC filtration step 625 in method 600, wherein the criteria for WBC noise cutoff was 20% of WBC samples having at least one anomalously methylated fragment overlapping the genomic region. The classifier labeled “v2” had a WBC noise cutoff at 5% of WBC samples having at least one anomalously methylated fragment overlapping the genomic region. The detection rate of the v0 classifier included 1.0% for non-cancer (effectively the false positive rate), 49% for all invasive cancers, 53.8% for solid cancers and multiple myeloma cancer, and 22.0% for lymphoid and myeloid cancers. The v1 classifier had detection rates of 0.8% for non-cancer, 53.2% for all invasive cancers, 59.6% for solid cancers and multiple myeloma cancer, and 14.4% for lymphoid and myeloid cancers. There is a tradeoff for the classifier's detection rate of lymphoid and myeloid cancers to bolster the detection rates for all invasive cancers and solid cancers and multiple myeloma cancer. The v2 classifier had detection rates of 1.2% for non-cancer, 51.9% for all invasive cancers, 57.4% for solid cancers and multiple myeloma cancer, and 19.5% for lymphoid and myeloid cancers. As with the v1 classifier, the v2 classifier also had improved detection rate for all invasive cancers and solid cancers and multiple myeloma cancer with a decreased detection rate of the lymphoid and myeloid cancers. The improved detection for the v2 classifier was less than the v1 classifier.

### V. Applications

### V.A. Early Detection of Cancer

In some embodiments, the methods and/or classifier of the present invention are used to detect the presence or absence of cancer in a subject suspected of having cancer. For example, a classifier (e.g., as described above in Section III and exampled in Section V) can be used to determine a cancer prediction describing a likelihood that a test feature vector is from a subject that has cancer.

In one embodiment, a cancer prediction is a likelihood (e.g., scored between 0 and 100) for whether the test sample has cancer (i.e. binary classification). Thus, the analytics system may determine a threshold for determining whether a test subject has cancer. For example, a cancer prediction of greater than or equal to 60 can indicate that the subject has cancer. In still other embodiments, a cancer prediction greater than or equal to 65, greater than or equal to 70, greater than or equal to 75, greater than or equal to 80, greater than or equal to 85, greater than or equal to 90, or greater than or equal to 95 indicates that the subject has cancer. In other embodiments, the cancer prediction can indicate the severity of disease. For example, a cancer prediction of 80 may indicate a more severe form, or later stage, of cancer compared to a cancer prediction below 80 (e.g., a probability score of 70). Similarly, an increase in the cancer prediction over time (e.g., determined by classifying test feature vectors from multiple samples from the same subject taken at two or more time points) can indicate disease progression or a decrease in the cancer prediction over time can indicate successful treatment.

In another embodiment, a cancer prediction comprises many prediction values, wherein each of a plurality of cancer types being classified (i.e. multiclass classification) for has a prediction value (e.g., scored between 0 and 100). The prediction values may correspond to a likelihood that a given training sample (and during inference, training sample) has each of the cancer types. The analytics system may identify the cancer type that has the highest prediction value and indicate that the test subject likely has that cancer type. In other embodiments, the analytics system further compares the highest prediction value to a threshold value (e.g., 50, 55, 60, 65, 70, 75, 80, 85, etc.) to determine that the test subject likely has that cancer type. In other embodiments, a prediction value can also indicate the severity of disease. For example, a prediction value greater than 80 may indicate a more severe form, or later stage, of cancer compared to a prediction value of 60. Similarly, an increase in the prediction value over time (e.g., determined by classifying test feature vectors from multiple samples from the same subject taken at two or more time points) can indicate disease progression or a decrease in the prediction value over time can indicate successful treatment.

According to aspects of the invention, the methods and systems of the present invention can be trained to detect or classify multiple cancer indications. For example, the methods, systems and classifiers of the present invention can be used to detect the presence of one or more, two or more, three or more, five or more, ten or more, fifteen or more, or twenty or more different types of cancer.

Examples of cancers that can be detected using the methods, systems and classifiers of the present invention include carcinoma, lymphoma, blastoma, sarcoma, and leukemia or lymphoid malignancies. More particular examples of such cancers include, but are not limited to, squamous cell cancer (e.g., epithelial squamous cell cancer), skin carcinoma, melanoma, lung cancer, including small-cell lung cancer, non-small cell lung cancer (“NSCLC”), adenocarcinoma of the lung and squamous carcinoma of the lung, cancer of the peritoneum, gastric or stomach cancer including gastrointestinal cancer, pancreatic cancer (e.g., pancreatic ductal adenocarcinoma), cervical cancer, ovarian cancer (e.g., high grade serous ovarian carcinoma), liver cancer (e.g., hepatocellular carcinoma (HCC)), hepatoma, hepatic carcinoma, bladder cancer (e.g., urothelial bladder cancer), testicular (germ cell tumor) cancer, breast cancer (e.g., HER2 positive, HER2 negative, and triple negative breast cancer), brain cancer (e.g., astrocytoma, glioma (e.g., glioblastoma)), colon cancer, rectal cancer, colorectal cancer, endometrial or uterine carcinoma, salivary gland carcinoma, kidney or renal cancer (e.g., renal cell carcinoma, nephroblastoma or Wilms' tumor), prostate cancer, vulval cancer, thyroid cancer, anal carcinoma, penile carcinoma, head and neck cancer, esophageal carcinoma, and nasopharyngeal carcinoma (NPC). Additional examples of cancers include, without limitation, retinoblastoma, thecoma, arrhenoblastoma, hematological malignancies, including but not limited to non-Hodgkin's lymphoma (NHL), multiple myeloma and acute hematological malignancies, endometriosis, fibrosarcoma, choriocarcinoma, laryngeal carcinomas, Kaposi's sarcoma, Schwannoma, oligodendroglioma, neuroblastomas, rhabdomyosarcoma, osteogenic sarcoma, leiomyosarcoma, and urinary tract carcinomas.

In some embodiments, the cancer is one or more of anorectal cancer, bladder cancer, breast cancer, cervical cancer, colorectal cancer, esophageal cancer, gastric cancer, head & neck cancer, hepatobiliary cancer, leukemia, lung cancer, lymphoma, melanoma, multiple myeloma, ovarian cancer, pancreatic cancer, prostate cancer, renal cancer, thyroid cancer, uterine cancer, or any combination thereof.

In some embodiments, the one or more cancer can be a “high-signal” cancer (defined as cancers with greater than 50% 5-year cancer-specific mortality), such as anorectal, colorectal, esophageal, head & neck, hepatobiliary, lung, ovarian, and pancreatic cancers, as well as lymphoma and multiple myeloma. High-signal cancers tend to be more aggressive and typically have an above-average cell-free nucleic acid concentration in test samples obtained from a patient.

### V.B. Cancer and Treatment Monitoring

In some embodiments, the cancer prediction can be assessed at multiple different time points (e.g., or before or after treatment) to monitor disease progression or to monitor treatment effectiveness (e.g., therapeutic efficacy). For example, the present invention include methods that involve obtaining a first sample (e.g., a first plasma cfDNA sample) from a cancer patient at a first time point, determining a first cancer prediction therefrom (as described herein), obtaining a second test sample (e.g., a second plasma cfDNA sample) from the cancer patient at a second time point, and determining a second cancer prediction therefrom (as described herein).

In certain embodiments, the first time point is before a cancer treatment (e.g., before a resection surgery or a therapeutic intervention), and the second time point is after a cancer treatment (e.g., after a resection surgery or therapeutic intervention), and the classifier is utilized to monitor the effectiveness of the treatment. For example, if the second cancer prediction decreases compared to the first cancer prediction, then the treatment is considered to have been successful. However, if the second cancer prediction increases compared to the first cancer prediction, then the treatment is considered to have not been successful. In other embodiments, both the first and second time points are before a cancer treatment (e.g., before a resection surgery or a therapeutic intervention). In still other embodiments, both the first and the second time points are after a cancer treatment (e.g., after a resection surgery or a therapeutic intervention). In still other embodiments, cfDNA samples may be obtained from a cancer patient at a first and second time point and analyzed. e.g., to monitor cancer progression, to determine if a cancer is in remission (e.g., after treatment), to monitor or detect residual disease or recurrence of disease, or to monitor treatment (e.g., therapeutic) efficacy.

Those of skill in the art will readily appreciate that test samples can be obtained from a cancer patient over any desired set of time points and analyzed in accordance with the methods of the invention to monitor a cancer state in the patient. In some embodiments, the first and second time points are separated by an amount of time that ranges from about 15 minutes up to about 30 years, such as about 30 minutes, such as about 1, 2, 3, 4, 5, 6, 7, 8, 9, 10, 11, 12, 13, 14, 15, 16, 17, 18, 19, 20, 21, 22, 23, or about 24 hours, such as about 1, 2, 3, 4, 5, 10, 15, 20, 25 or about 50 days, or such as about 1, 2, 3, 4, 5, 6, 7, 8, 9, 10, 11, or 12 months, or such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or about 30 years. In other embodiments, test samples can be obtained from the patient at least once every 5 months, at least once every 6 months, at least once a year, at least once every 2 years, at least once every 3 years, at least once every 4 years, or at least once every 5 years.

### V.C. Treatment

In still another embodiment, the cancer prediction can be used to make or influence a clinical decision (e.g., diagnosis of cancer, treatment selection, assessment of treatment effectiveness, etc.). For example, in one embodiment, if the cancer prediction (e.g., for cancer or for a particular cancer type) exceeds a threshold, a physician and/or the analytics system can prescribe an appropriate treatment (e.g., a resection surgery, radiation therapy, chemotherapy, and/or immunotherapy). In other embodiments, the analytics system may provide treatment recommendations based on the cancer prediction to a physician to work with the patient to define a treatment plan.

A classifier (as described herein) can be used to determine a cancer prediction that a sample feature vector is from a subject that has cancer. In one embodiment, an appropriate treatment (e.g., resection surgery or therapeutic) is prescribed when the cancer prediction exceeds a threshold. For example, in one embodiment, if the cancer prediction is greater than or equal to 60 one or more appropriate treatments are prescribed. In another embodiment, if the cancer prediction is greater than or equal to 65, greater than or equal to 70, greater than or equal to 75, greater than or equal to 80, greater than or equal to 85, greater than or equal to 90, or greater than or equal to 95, one or more appropriate treatments are prescribed. In other embodiments, the cancer prediction can indicate the severity of disease. An appropriate treatment matching the severity of the disease may then be prescribed.

In some embodiments, the treatment is one or more cancer therapeutic agents selected from the group consisting of a chemotherapy agent, a targeted cancer therapy agent, a differentiating therapy agent, a hormone therapy agent, and an immunotherapy agent. For example, the treatment can be one or more chemotherapy agents selected from the group consisting of alkylating agents, antimetabolites, anthracyclines, anti-tumor antibiotics, cytoskeletal disruptors (taxans), topoisomerase inhibitors, mitotic inhibitors, corticosteroids, kinase inhibitors, nucleotide analogs, platinum-based agents and any combination thereof. In some embodiments, the treatment is one or more targeted cancer therapy agents selected from the group consisting of signal transduction inhibitors (e.g. tyrosine kinase and growth factor receptor inhibitors), histone deacetylase (HDAC) inhibitors, retinoic receptor agonists, proteosome inhibitors, angiogenesis inhibitors, and monoclonal antibody conjugates. In some embodiments, the treatment is one or more differentiating therapy agents including retinoids, such as tretinoin, alitretinoin and bexarotene. In some embodiments, the treatment is one or more hormone therapy agents selected from the group consisting of anti-estrogens, aromatase inhibitors, progestins, estrogens, anti-androgens, and GnRH agonists or analogs. In one embodiment, the treatment is one or more immunotherapy agents selected from the group comprising monoclonal antibody therapies such as rituximab (RITUXAN) and alemtuzumab (CAMPATH), non-specific immunotherapies and adjuvants, such as BCG, interleukin-2 (IL-2), and interferon-alfa, immunomodulating drugs, for instance, thalidomide and lenalidomide (REVLIMID). It is within the capabilities of a skilled physician or oncologist to select an appropriate cancer therapeutic agent based on characteristics such as the type of tumor, cancer stage, previous exposure to cancer treatment or therapeutic agent, and other characteristics of the cancer.

### VI. Kit Implementation

Also disclosed herein are kits for performing the methods described above including the methods relating to the cancer classifier. The kits may include one or more collection vessels for collecting a sample from the individual comprising genetic material. The sample can include blood, plasma, serum, urine, fecal, saliva, other types of bodily fluids, or any combination thereof. Such kits can include reagents for isolating nucleic acids from the sample. The reagents can further include reagents for sequencing the nucleic acids including buffers and detection agents. In one or more embodiments, the kits may include one or more sequencing panels comprising probes for targeting particular genomic regions, particular mutations, particular genetic variants, or some combination thereof. In other embodiments, samples collected via the kit are provided to a sequencing laboratory that may use the sequencing panels to sequence the nucleic acids in the sample.

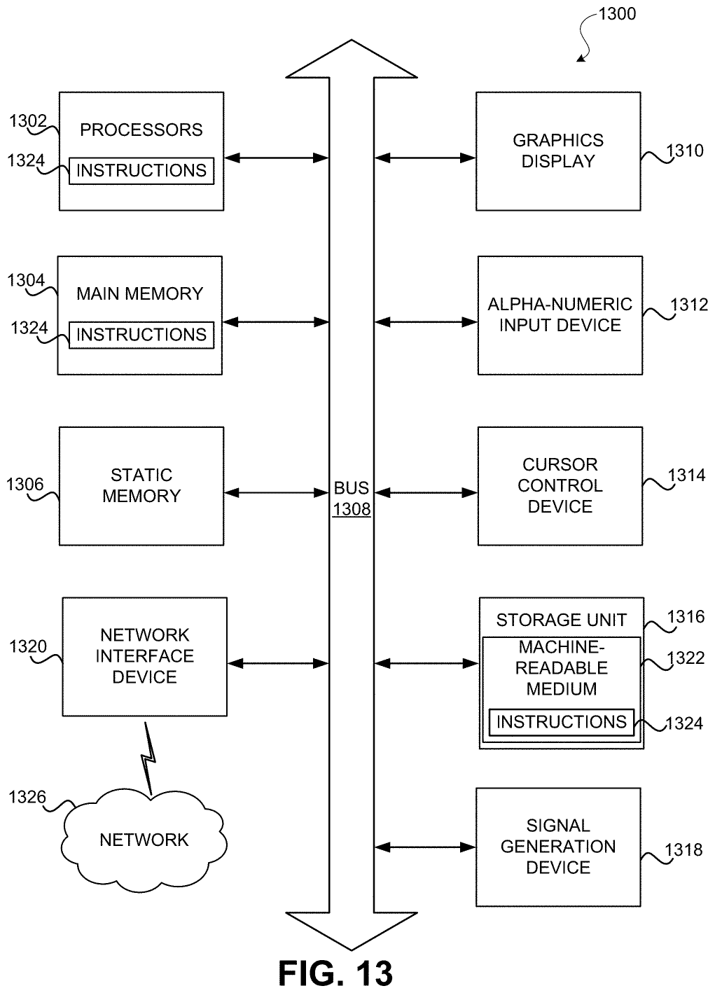

A kit can further include instructions for use of the reagents included in the kit. For example, a kit can include instructions for collecting the sample, extracting the nucleic acid from the test sample. Example instructions can be the order in which reagents are to be added, centrifugal speeds to be used to isolate nucleic acids from the test sample, how to amplify nucleic acids, how to sequence nucleic acids, or any combination thereof. The instructions may further illumine how to operate a computing device (e.g., the computer system 1300 of FIG. 13) as the analytics system 200 of FIGS. 2A & 2B, for the purposes of performing any of the methods described throughout.

In addition to the above components, the kit may include computer-readable storage media storing computer software for performing the various methods described throughout the disclosure. One form in which these instructions can be present is as printed information on a suitable medium or substrate, e.g., a piece or pieces of paper on which the information is printed, in the packaging of the kit, in a package insert. Yet another means would be a computer readable medium, e.g., diskette, CD, hard-drive, network data storage, on which the instructions have been stored in the form of computer code. Yet another means that can be present is a website address which can be used via the internet to access the information at a removed site.

### VII. Computing Machine Architecture

FIG. 13 shows a schematic of an example computer system 1300 for implementing various methods of the present invention. In particular, FIG. 13 is a block diagram illustrating components of an example computing machine that is capable of reading instructions from a computer-readable medium and execute them in a processor (or controller). A computer described herein can include a single computing machine shown in FIG. 13, a virtual machine, a distributed computing system that includes multiples nodes of computing machines shown in FIG. 13, or any other suitable arrangement of computing devices.

By way of example, FIG. 13 shows a diagrammatic representation of a computing machine in the example form of a computer system 1300 within which instructions 1324 (e.g., software, program code, or machine code), which can be stored in a computer-readable medium for causing the machine to perform any one or more of the processes discussed herein can be executed. In some embodiments, the computing machine operates as a standalone device or can be connected (e.g., networked) to other machines. In a networked deployment, the machine can operate in the capacity of a server machine or a client machine in a server-client network environment, or as a peer machine in a peer-to-peer (or distributed) network environment.

The structure of a computing machine described in FIG. 13 can correspond to any software, hardware, or combined components, including but not limited to any engines, modules, computing server, machines that are used to perform one or more processes described herein. While FIG. 13 shows various hardware and software elements, each of the components described herein can include additional or fewer elements.

By way of example, a computing machine can be a personal computer (PC), a tablet PC, a set-top box (STB), a personal digital assistant (PDA), a cellular telephone, a smartphone, a web appliance, a network router, an internet of things (IoT) device, a switch or bridge, or any machine capable of executing instructions 1324 that specify actions to be taken by that machine. Further, while only a single machine is illustrated, the term “machine” and “computer” can also be taken to include any collection of machines that individually or jointly execute instructions 1324 to perform any one or more of the methodologies discussed herein.

The example computer system 1300 includes one or more processors 1302 such as a CPU (central processing unit), a GPU (graphics processing unit), a TPU (tensor processing unit), a DSP (digital signal processor), a system on a chip (SOC), a controller, a state equipment, an application-specific integrated circuit (ASIC), a field-programmable gate array (FPGA), or any combination of these. Parts of the computing system 1300 can also include a memory 1304 that store computer code including instructions 1324 that can cause the processors 1302 to perform certain actions when the instructions are executed, directly or indirectly by the processors 1302. Instructions can be any directions, commands, or orders that can be stored in different forms, such as equipment-readable instructions, programming instructions including source code, and other communication signals and orders. Instructions can be used in a general sense and are not limited to machine-readable codes.

One and more methods described herein improve the operation speed of the processors 1302 and reduces the space required for the memory 1304. For example, the machine learning methods described herein reduces the complexity of the computation of the processors 1302 by applying one or more novel techniques that simplify the steps in training, reaching convergence, and generating results of the processors 1302. The algorithms described herein also reduces the size of the models and datasets to reduce the storage space requirement for memory 1304.

The performance of certain of the operations can be distributed among the more than processors, not only residing within a single machine, but deployed across a number of machines. In some example embodiments, the one or more processors or processor-implemented modules can be located in a single geographic location (e.g., within a home environment, an office environment, or a server farm). In other example embodiments, the one or more processors or processor-implemented modules can be distributed across a number of geographic locations. Even though in the specification or the claims may refer some processes to be performed by a processor, this should be construed to include a joint operation of multiple distributed processors.

The computer system 1300 can include a main memory 1304, and a static memory 1306, which are configured to communicate with each other via a bus 1308. The computer system 1300 can further include a graphics display unit 1310 (e.g., a plasma display panel (PDP), a liquid crystal display (LCD), a projector, or a cathode ray tube (CRT)). The graphics display unit 1310, controlled by the processors 1302, displays a graphical user interface (GUI) to display one or more results and data generated by the processes described herein. The computer system 1300 can also include alphanumeric input device 1312 (e.g., a keyboard), a cursor control device 1314 (e.g., a mouse, a trackball, a joystick, a motion sensor, or other pointing instrument), a storage unit 1316 (a hard drive, a solid state drive, a hybrid drive, a memory disk, etc.), a signal generation device 1318 (e.g., a speaker), and a network interface device 1320, which also are configured to communicate via the bus 1308.

The storage unit 1316 includes a computer-readable medium 1322 on which is stored instructions 1324 embodying any one or more of the methodologies or functions described herein. The instructions 1324 can also reside, completely or at least partially, within the main memory 1304 or within the processor 1302 (e.g., within a processor's cache memory) during execution thereof by the computer system 1300, the main memory 1304 and the processor 1302 also constituting computer-readable media. The instructions 1324 can be transmitted or received over a network 1326 via the network interface device 1320.

While computer-readable medium 1322 is shown in an example embodiment to be a single medium, the term “computer-readable medium” should be taken to include a single medium or multiple media (e.g., a centralized or distributed database, or associated caches and servers) able to store instructions (e.g., instructions 1324). The computer-readable medium can include any medium that is capable of storing instructions (e.g., instructions 1324) for execution by the processors (e.g., processors 1302) and that cause the processors to perform any one or more of the methodologies disclosed herein. The computer-readable medium can include, but not be limited to, data repositories in the form of solid-state memories, optical media, and magnetic media. The computer-readable medium does not include a transitory medium such as a propagating signal or a carrier wave.

### VIII. Additional Considerations

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules can be embodied in software, firmware, hardware, or any combinations thereof.

Any of the steps, operations, or processes described herein can be performed or implemented with one or more hardware or software modules, alone or in combination with other devices. In one embodiment, a software module is implemented with a computer program product including a computer-readable non-transitory medium containing computer program code, which can be executed by a computer processor for performing any or all of the steps, operations, or processes described.

Embodiments of the invention can also relate to a product that is produced by a computing process described herein. Such a product can include information resulting from a computing process, where the information is stored on a non-transitory, tangible computer readable storage medium and can include any embodiment of a computer program product or other data combination described herein.

Finally, the language used in the specification has been principally selected for readability and instructional purposes, but one of ordinary skill in the art would recognize the applicability of the principles herein to other contexts and applications. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

1.-20. (canceled)

21. A method for training a cancer classifier, the method comprising:
for each of a plurality of training samples comprising cancer samples and non-cancer samples, each training sample comprising a plurality of methylation sequence reads including methylation information of cell-free DNA fragments:
for each methylation sequence read, applying a probabilistic noise model, corresponding to a genomic region of a plurality of genomics regions that the methylation sequence read overlaps with, to the methylation sequence read to determine an anomaly score indicating a likelihood of observing the methylation pattern in healthy samples, wherein each probabilistic noise model is trained with methylation sequence reads from healthy samples;
determining a feature vector comprising a feature for each genomic region based on a count of methylation sequence reads overlapping the genomic region with an anomaly score below a threshold anomaly score; and
training the cancer classifier with the feature vectors of the training samples to determine a cancer prediction based on an input feature vector.

22. The method of claim 22, wherein each probabilistic noise model is parametrized by a mean and a dispersion of a measure of methylated CpG sites in methylation sequence reads from the healthy samples.

23. The method of claim 22, wherein each probabilistic noise model is trained by:
determining posterior distributions of the mean and the dispersion for each genomic region of the plurality of genomic regions using a Bayesian inference, wherein the Bayesian inference is determined using Markov chain Monte Carlo.

24. The method of claim 23, wherein the posterior distributions are beta binomial distributions.

25. The method of claim 21, wherein the anomaly score determined by the trained probabilistic noise models for each methylation sequence read is based on a p-value for the methylation sequence read indicating a probability that the methylation sequence read is anomalously methylated.

26. The method of claim 25, wherein the anomaly score for each methylation sequence read is the p-value for the methylation sequence read.

27. The method of claim 25, wherein the anomaly score for each methylation sequence read is determined by applying a transformation to the p-value determined for the methylation sequence read.

28. The method of claim 27, wherein the transformation is a logarithmic or nonlinear function.

29. The method of claim 21, wherein a first genomic region of the plurality of genomic regions is associated with a first mean and a first dispersion, and wherein a second genomic region of the plurality of genomic regions is associated with a second mean and a second dispersion different than the first mean and the first dispersion, respectively.

30. The method of claim 21, wherein a first genomic region of the plurality of genomic regions includes a first number of CpG sites, and the second genomic region of the plurality of genomic regions includes a second number of CpG sites, that is different than the first number of CpG sites.

31. The method of claim 21, further comprising:
for each White Blood Cell (WBC) sample of a plurality of WBC samples, determining an anomaly score for each of a plurality of methylation sequence reads from the WBC sample by applying the trained probabilistic noise model associated with the genomic region that the methylation sequence read overlaps with;
for each WBC sample, determining a count of anomalously methylated fragments in each genomic region of the plurality of genomic regions by comparing the anomaly scores of the methylation sequence reads with a threshold anomaly score; and
for each genomic region of the plurality of genomic regions, labeling the genomic region as noisy if there is more than a threshold percentage of WBC samples with a threshold number of anomalously methylated fragments overlapping the genomic region.

32. The method of claim 31, further comprising:
excluding the genomic regions labeled as noisy from use in the training of the classifier, wherein the feature vectors generated for the training samples exclude the ratios of the genomic regions labeled as noisy.

33. The method of claim 31, further comprising:
assigning a default weight to each genomic region of the plurality of genomic regions;
reassigning a first weight to the genomic regions labeled as noisy, wherein the first weight is lower than the default weight; and
for each training sample, multiplying each ratio of the feature vector with the corresponding weight for the genomic region associated with the ratio.

34. The method of claim 31, wherein the threshold percentage is selected from the range of 5% to 40%.

35. The method of claim 31, wherein the threshold number of anomalously methylated fragments is selected from the range of 1-10.

36. A method for predicting cancer status of a test sample comprising a plurality of methylation sequence reads including methylation information of cell-free DNA fragments, the method comprising:
for each methylation sequence read, applying a probabilistic noise model, corresponding to a genomic region of a plurality of genomics regions that the methylation sequence read overlaps with, to the methylation sequence read to determine an anomaly score indicating a likelihood of observing the methylation pattern in healthy samples, wherein each probabilistic noise model is trained with methylation sequence reads from healthy samples;
determining a feature vector comprising a feature for each genomic region based on a count of methylation sequence reads overlapping the genomic region with an anomaly score below a threshold anomaly score; and
applying a cancer classifier to the feature vector to determine a cancer prediction.

37. The method of claim 36, wherein the cancer classifier is trained by:
for each of a plurality of training samples comprising cancer samples and non-cancer samples, each training sample comprising a plurality of methylation sequence reads including methylation information of cell-free DNA fragments:
for each methylation sequence read, applying the probabilistic noise model, corresponding to the genomic region of the plurality of genomics regions that the methylation sequence read overlaps with, to the methylation sequence read to determine an anomaly score indicating a likelihood of observing the methylation pattern in healthy samples, wherein each probabilistic noise model is trained with methylation sequence reads from healthy samples;
determining a feature vector comprising a feature for each genomic region based on a count of methylation sequence reads overlapping the genomic region with an anomaly score below the threshold anomaly score; and
training the cancer classifier with the feature vectors of the training samples to determine a cancer prediction based on an input feature vector.

38. The method of claim 36, wherein the cancer prediction estimates a tumor fraction of the test sample.

39. The method of claim 36, wherein the cancer prediction indicates a presence of a disease state in the test sample.

40. The method of claim 39, wherein the disease state is selected from the group consisting of breast cancer, uterine cancer, cervical cancer, ovarian cancer, bladder cancer, urothelial cancer of renal pelvis, renal cancer other than urothelial, prostate cancer, anorectal cancer, colorectal cancer, esophageal cancer, gastric cancer, hepatobiliary cancer arising from hepatocytes, hepatobiliary cancer arising from cells other than hepatocytes, pancreatic cancer, squamous cell cancer of the upper gastrointestinal tract, upper gastrointestinal cancer other than squamous, head and neck cancer, lung cancer, lung adenocarcinoma, small cell lung cancer, squamous cell lung cancer and cancer other than adenocarcinoma or small cell lung cancer, neuroendocrine cancer, melanoma, thyroid cancer, sarcoma, multiple myeloma, lymphoma, and leukemia, and other hematological conditions.

41. The method of claim 36, wherein the cancer prediction indicates a stage of cancer present in the test sample.

42. The method of claim 36, further comprising:
returning the cancer prediction with treatment recommendation based on the cancer prediction.

43.-61. (canceled)
